(19) United States
(12) Patent Application Publication
(10) Pub. No.: US 2016/0287667 A1
(43) Pub. Date: Oct. 6, 2016

(54) METHODS AND COMPOSITIONS FOR REJUVENATING NEUROMUSCULAR JUNCTIONS

(71) Applicant: PRESIDENT AND FELLOWS OF HARVARD COLLEGE, Cambridge, MA (US)

(72) Inventor: Amy J. Wagers, Cambridge, MA (US)

(21) Appl. No.: 15/035,331

(22) PCT Filed: Nov. 7, 2014
(86) PCT No.: PCT/US14/64648

§ 371 Date: May 9, 2016

Related U.S. Application Data
(60) Provisional application No. 61/901,875, filed on Nov. 8, 2013.

(51) Int. Cl. A61K 38/18; A61K 39/395; A61K 9/00; A61K 45/06; C07K 16/22; A61K 47/48

(52) U.S. Cl. CPC A61K 38/1875; C07K 16/22; A61K 47/48284; A61K 47/48215; A61K 9/0019; A61K 45/06; A61K 39/3955; A61K 2039/505

(57) ABSTRACT

The disclosure relates to methods and compositions for rejuvenating neuromuscular junctions, and treating, preventing, or delaying the onset of, neuromuscular junction fragmentation and related disorders, neuromuscular junction degeneration and related disorders, motor neuron degeneration and related disorders, skeletal muscle conditions (e.g., muscle atrophy), and neuromuscular diseases (e.g., amyotrophic lateral sclerosis).

### RELATED APPLICATIONS

This application claims the benefit of U.S. Provisional Application No. 61/901,875, filed Nov. 8, 2013, the entire teachings of which are incorporated herein by reference.

### GOVERNMENT SUPPORT

This invention was made with government support under UO1 HL100402 and RO1 AG033053 awarded by the National Institutes of Health. The government has certain rights in the invention.

### BACKGROUND OF THE INVENTION

Age-dependent dysfunction in adult cells is attributable to both cell-intrinsic and -extrinsic inputs. Critical mechanisms underlying the functional decline of aged cells remain elusive. Accordingly, there exists a need to identify factors that are able to promote or reverse age-associated changes in tissues as diverse as the skeletal muscle, liver and CNS (Wagers and Conboy, Cell 2005; 122, 659; Ruckh et al. Cell Stem Cell 2005; 10, 96).

### SUMMARY OF THE INVENTION

The methods and compositions described herein are useful for rejuvenating neuromuscular junctions, treating, preventing, or delaying the onset of, neuromuscular junction degeneration, and treating, preventing, or delaying the onset of neuromuscular disease (e.g., amyotrophic lateral sclerosis).

In an aspect, the present invention provides a method of rejuvenating neuromuscular junctions in a subject in need thereof, the method comprising administering to the subject a composition which increases the level of GDF11 polypeptide in the subject.

In an aspect, the present invention provides a method of treating, preventing, or delaying the onset of, neuromuscular junction fragmentation or a related disorder in a subject in need thereof, the method comprising administering to the subject an effective amount of a composition which increases the level of GDF11 polypeptide in the subject.

In an aspect, the present invention provides a method of treating, preventing, or delaying the onset of, neuromuscular junction degeneration or a related disorder in a subject in need thereof, the method comprising administering to the subject an effective amount of a composition which increases the level of GDF11 polypeptide in the subject.

In an aspect, the present invention provides a method of treating, preventing, or delaying the onset of, motor neuron degeneration or a related disorder in a subject in need thereof, the method comprising administering to the subject an effective amount of a composition which increases the level of GDF11 polypeptide in the subject.

In an aspect, the present invention provides a method of treating, preventing, or delaying the onset of, muscle atrophy in a subject in need thereof, the method comprising administering to the subject an effective amount of a composition which increases the level of GDF11 polypeptide in the subject.

In an aspect, the present invention provides a method of treating, preventing, or delaying the onset of, a neuromuscular disease in a subject in need thereof, the method comprising administering to the subject an effective amount of a composition which increases the level of GDF11 polypeptide in the subject.

In an aspect, the present invention provides a method of treating, preventing, or delaying the onset of amyotrophic lateral sclerosis in a subject in need thereof, the method comprising administering to the subject an effective amount of a composition which increases the level of GDF11 polypeptide in the subject.

In an aspect, the present invention relates to the use of a composition comprising a GDF11 polypeptide or functional fragment or variant thereof for rejuvenating neuromuscular junctions in a subject in need thereof, wherein increased levels of the GDF11 polypeptide or functional fragment or variant thereof in the subject rejuvenate neuromuscular junctions in the subject.

In an aspect, the present invention relates to the use of a composition comprising a GDF11 polypeptide or a functional fragment or variant thereof for treating, preventing, or delaying the onset of, neuromuscular junction fragmentation or a related disorder in a subject in need thereof, wherein increased levels of the GDF11 polypeptide or functional fragment or variant thereof reverses or reduces neuromuscular junction fragmentation in the subject, thereby treating, preventing, or delaying the onset of, neuromuscular junction fragmentation or the related disorder in the subject.

In an aspect, the present invention relates to the use of a composition comprising a GDF11 polypeptide or a functional fragment or variant thereof for treating, preventing, or delaying the onset of, neuromuscular junction degeneration or a related disorder in a subject in need thereof, wherein increased levels of the GDF11 polypeptide or functional fragment or variant thereof in the subject treat, prevent, or delay the onset of, neuromuscular junction degeneration or the related disorder in the subject.

In an aspect, the present invention relates to the use of a composition comprising a GDF11 polypeptide or a functional fragment or variant thereof for treating, preventing, or delaying the onset of, motor neuron degeneration or a related disorder in a subject in need thereof, wherein increased levels of the GDF11 polypeptide or functional fragment or variant thereof in the subject treat, prevent, or delay the onset of, motor neuron degeneration or the related disorder in the subject.

In an aspect, the present invention relates to the use of a composition comprising a GDF11 polypeptide or a functional fragment or variant thereof for treating, preventing, or delaying the onset of, muscle atrophy in a subject in need thereof, wherein increased levels of the GDF11 polypeptide or functional fragment or variant thereof in the subject treat, prevent, or delay the onset of, muscle atrophy in the subject.

In an aspect, the present invention relates to the use of a composition comprising a GDF11 polypeptide or a functional fragment or variant thereof for treating, preventing, or delaying the onset of, amyotrophic lateral sclerosis in a subject in need thereof, wherein increased levels of the GDF11 polypeptide or functional fragment or variant thereof in the subject treat, prevent, or delay the onset of, amyotrophic lateral sclerosis in the subject.

In certain embodiments, the composition causes one or more of a decrease in fragmentation of neuromuscular junctions, an increase or maintenance of neuromuscular innervation, a decrease of neuromuscular denervation, preservation or restoration of motor units, an increase in the size of postsynaptic endplates, an increase in the number of postsynaptic endplates, an increase in the length of postsynaptic endplates, an increase in the density of postsynaptic folds, normalization of neuromuscular junction morphology, a decrease in denervation of fast-twitch fibers, an increase in the number of nerve terminal branches per endplate, an increase in the number of terminal sprouts, an increase or maintenance of synaptic vesicles, mitochondrial content, or nerve terminal area in presynaptic terminals, reversal of age-related changes in the quantal content of neurotransmitter release measured by decreases in amplitude of evoked endplate potentials (EPP), a reversal of age-related decline in axonal transport, preservation or restoration of muscle fibers, an increase in acetylcholine receptor number, a decrease in partially innervated or completed denervated neuromuscular junctions, an increase in the number of functional motor units in fast twitch muscle, enhanced neuromuscular recovery, enhanced physical performance, an increase in neuromuscular junction area, an increase in muscle size, and any combination thereof.

In certain embodiments, the subject has been diagnosed with a condition, disease, or disorder associated with aging. In certain embodiments, the condition, disease, or disorder associated with aging is selected from the group consisting of a skeletal muscle condition, a neuromuscular disease, a neurodegenerative disorder, a neuromuscular junction disease, and combinations thereof.

In certain embodiments, the level of GDF11 polypeptide is increased in the systemic circulation of the subject. In certain embodiments, the level of GDF11 polypeptide is increased in the skeletal muscle tissue of the subject.

In certain embodiments, the composition comprises an agonist antibody that increases expression or activity of GDF11 polypeptide in the subject. In certain embodiments, the composition comprises an isolated or recombinant GDF11 polypeptide. In certain embodiments, the composition comprises a GDF11 polypeptide comprising the amino acid sequence of SEQ ID NO: 3. In certain embodiments, the composition comprises a GDF11 polypeptide comprising the amino acid sequence of SEQ ID NO: 2. In certain embodiments, the composition comprises a GDF11 polypeptide comprising the amino acid sequence of SEQ ID NO: 1. In certain embodiments, the composition comprises a GDF11 polypeptide comprising the amino acid sequence of SEQ ID NO: 4. In certain embodiments, the composition comprises homodimers of GDF11 polypeptides comprising the amino acid sequence of any of SEQ ID NOs: 1, 2, 3 or 4. In certain embodiments, the composition comprises complexes of GDF11 polypeptides comprising the amino acid sequence of any of SEQ ID NOs: 1, 2, 3 or 4. In certain embodiments, the composition comprises a nucleic acid encoding a GDF11 polypeptide or a functional fragment or variant thereof.

In certain embodiments, the GDF11 polypeptide comprises a modified GDF11 polypeptide. In certain embodiments, the modified GDF11 polypeptide comprises a modification selected from the group consisting of fusion to an Fc fragment, pegylation, conjugation to albumin, an amino acid mutation that prevents or reduces proteolytic degradation, an amino acid mutation that prolongs half-life, and any combination thereof.

In certain embodiments, the composition is administered via a route selected from the group consisting of intravenously, subcutaneously, intra-arterially, intra-muscularly, and intrathecally.

In certain embodiments, the level of GDF11 polypeptide is increased by at least 100%. In certain embodiments, the level of GDF11 polypeptide is increased to at least 75% of a healthy reference level.

In an aspect, the present invention provides, a pharmaceutical composition comprising a GDF11 polypeptide or a functional fragment or variant thereof, and a pharmaceutically acceptable carrier. In certain embodiments, the pharmaceutical composition includes an agent that promotes survival and maintenance of presynaptic and postsynaptic apparatus at the neuromuscular junction. In certain embodiments, the agent is selected from the group consisting of a neurotrophic factor, a myotrophic factor, a myogenic regulatory factor, and combinations thereof.

In certain embodiments, the inventions disclosed herein increase the concentration or production of endogenous GDF11 in a subject. For example, in certain aspects, the concentration of GDF11 polypeptide in a subject (e.g., the concentration of GDF11 in the skeletal muscle of a subject) may be increased by exercise. Accordingly, in certain embodiments the inventions disclosed herein relate to methods of rejuvenating neuromuscular junctions in a subject in need thereof, comprising increasing the level of GDF11 polypeptide in the subject by exposing such subject to an exercise regimen. Also disclosed are methods of treating, preventing, or delaying the onset of, neuromuscular junction fragmentation or a related disorder in a subject in need thereof, the method comprising increasing the level of GDF11 polypeptide in the subject by exposing such subject to an exercise regimen (e.g., an exercise regimen capable of building the size or strength of the subject's muscle tissue). Similarly, also disclosed are methods of treating, preventing, or delaying the onset of, muscle atrophy, neuromuscular junction degeneration, motor neuron degeneration or a neuromuscular disease (e.g., amyotrophic lateral sclerosis) in a subject in need thereof, the method comprising increasing the level of GDF11 polypeptide in the subject by exposing such subject to an exercise regimen.

In certain embodiments, a subject's levels or concentrations of GDF11 polypeptide (e.g., the concentration of GDF11 polypeptide in the skeletal muscle of a subject) may be increased by exercise and the administration of a high fat diet to the subject. In certain aspects, disclosed herein are methods of rejuvenating neuromuscular junctions in a subject in need thereof, comprising increasing the level of GDF11 polypeptide in the subject by exposing such subject to an exercise regimen and a high fat diet. Also disclosed are methods of treating, preventing, or delaying the onset of neuromuscular junction fragmentation or a related disorder in a subject in need thereof, the method comprising increasing the level of GDF11 polypeptide in the subject by exposing such subject to an exercise regimen and a high fat diet. Similarly, also disclosed are methods of treating, preventing, or delaying the onset of muscle atrophy, neuromuscular junction degeneration, motor neuron degeneration or a neuromuscular disease (e.g., amyotrophic lateral sclerosis) in a subject in need thereof, the method comprising increasing the level of GDF11 polypeptide in the subject by exposing such subject to an exercise regimen (e.g., an exercise regimen capable of building the size or strength of the subject's muscle tissue) and a high fat diet.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a comparison between a normal and fragmented (i.e., exhibits four or more islands) neuromuscular junctions.

FIGS. 2A, 2B, 2C and 2D demonstrate the improved morphology of neuromuscular junctions (NMJs) in aged mice exposed to heterochronic parabiosis. FIGS. 2A, 2B, and 2C are immunostains showing neuromuscular junction morphology of isochronically joined young mice (Iso-Young; n=4-8; 70 NMJs per mouse; FIG. 2A), isochronically joined old mice (iso-Old; n=4-8; 70 NMJs per mouse; FIG. 2B), and heterochronically joined young-old mice (Het-Old; n=4-8; 70 NMJs per mouse; FIG. 2C). FIG. 2D is a bar graph quantifying the percentage of fragmentation of the Iso-Young, Iso-Old, and Het-Old joined mice neuromuscular junctions shown in FIGS. 2A, 2B, and 2C, respectively.

FIG. 3 is a bar graph demonstrating that GDF11 repairs neuromuscular junction fragmentation in aged mice treated with GDF11.

FIG. 4 is a graph demonstrating improved physical performance, as measured by the distance run until exhaustion, in a subset of aged mice treated with recombinant GDF11.

FIGS. 5A, 5B and 5C are electron micrographs demonstrating improved muscle ultrastructure in aged mice exposed to heterochronic parabiosis. FIG. 5A is an electron micrograph showing muscle ultrastructure of young isochronic mice compared to old isochronic (FIG. 5B) and old heterochronic (FIG. 5C), illustrating reduced intramyofibrillar lipid accumulation (*), mitochondrial swelling and vacuolization, and tubular aggregation (TA).

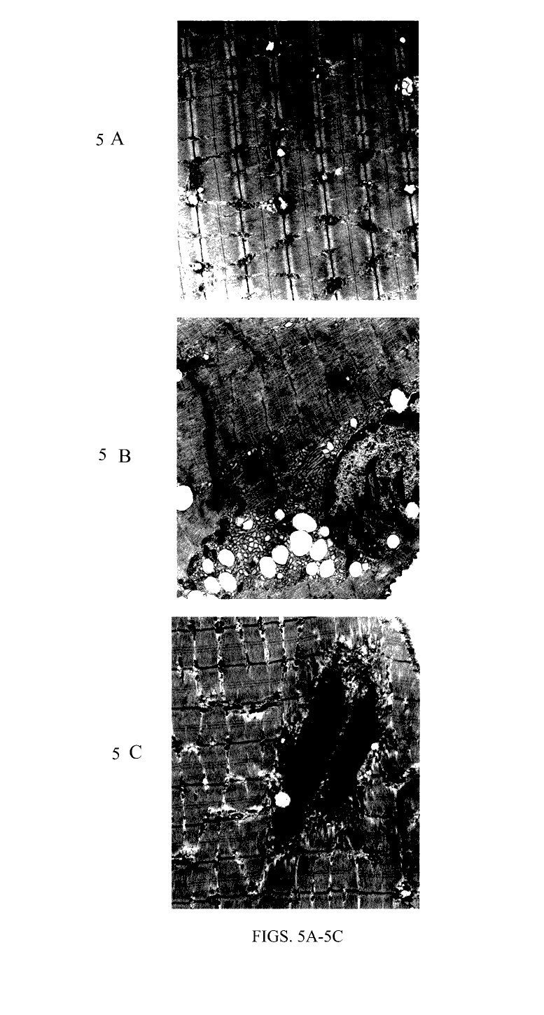

FIGS. 6A and 6B are electron micrographs demonstrating the effect of GDF11 supplementation on muscle histology. FIG. 6A shows longitudinal myofibril sections of untreated 24 month old aged mice (control) displaying muscle fiber atrophy and necrosis typically associated with degeneration, including loss of striations, decreased relative uniformity in muscle fiber size and shape, increased separation between adjacent myofibrils, and disruption of the sarcomeres. In contrast, as is shown in FIG. 6B, the muscle ultrastructure of the GDF11 treated mice shows that adjacent myofibrils are relatively parallel resulting in the overall repeated cross-striations of the myofibers, which are relatively uniform in size and shape. In addition, the A and I bands and Z lines are clearly visible in the myofibrils, which consist of regularly repeating series of sarcomeres.

FIGS. 7A, 7B, 7C and 7D demonstrate the effect of GDF11 supplementation on muscle ultrastructure and neuromuscular junctions. FIG. 7A is a micrograph cross section of skeletal muscle from a 2 month old mouse (young control), demonstrating normal morphology that consists of tightly packed polygonal fibers of relatively uniform size, with a small amount (˜5%) of extracellular material. FIG. 7B is a micrograph cross section of skeletal muscle from a 24 month old mouse (aged control), demonstrating fibrotic morphology in which the extracellular material is increased to ˜20% of the cross section, fibers are loosely packed, extracellular space is hypercellular, and fiber sizes are highly variable. FIG. 7C is a micrograph cross section of skeletal muscle from a 24 month old mouse treated with GDF11 (GDF11 treated aged), demonstrating a reversal of fibrotic morphology as evidenced by a decrease in the extracellular material, more tightly packed fibers, decreased extracellular space, and increased uniformity of fiber sizes. FIG. 7D is a bar graph quantifying muscle fiber size, as measured by cross sectional area (μm²), of the young control, aged control, and GDF11 treated aged mice shown in FIGS. 7A, 7B, and 7C, respectively.

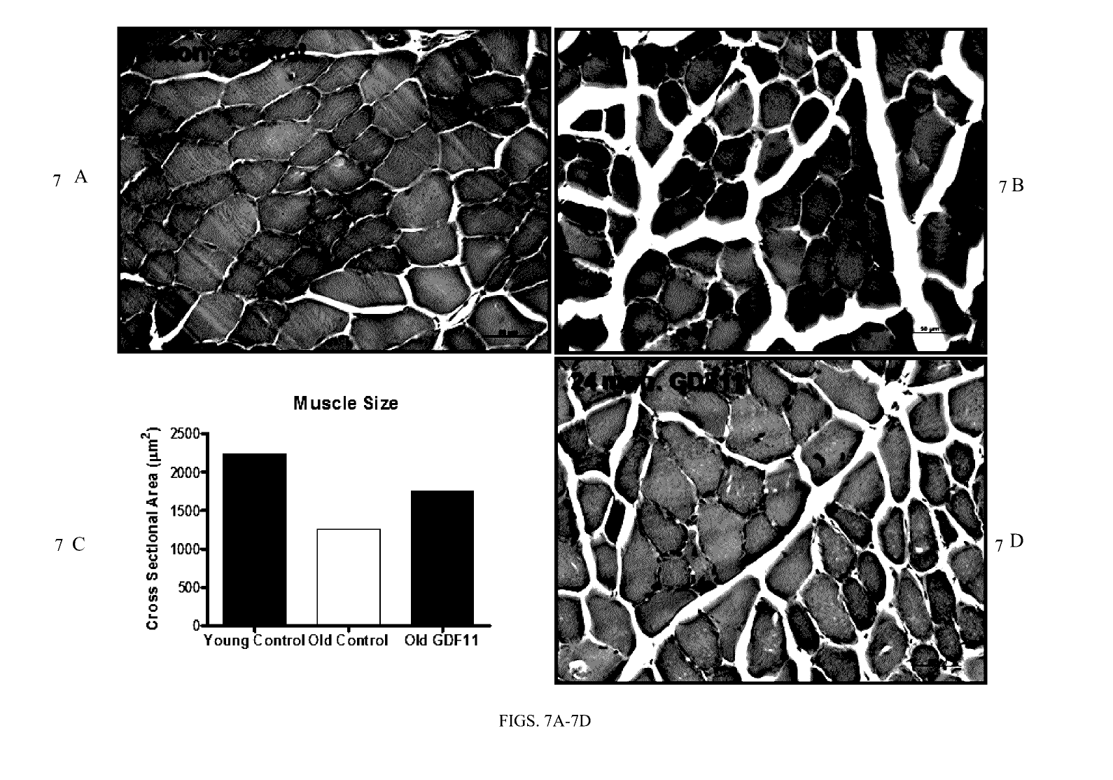

FIGS. 8A, 8B, 8C and 8D demonstrate the effect of GDF11 supplementation on neuromuscular junctions. FIGS. 8A, 8B, and 8C are immunostains showing neuromuscular junction morphology of Young (5 month old) mice, Old (24 month old) mice, and Old (24 month old) mice treated with GDF11. FIG. 8D is a bar graph quantifying the area of neuromuscular junctions in Young, Old, and GDF11-treated Old, according to the immunostains shown in FIGS. 8A, 8B, and 8C, respectively.

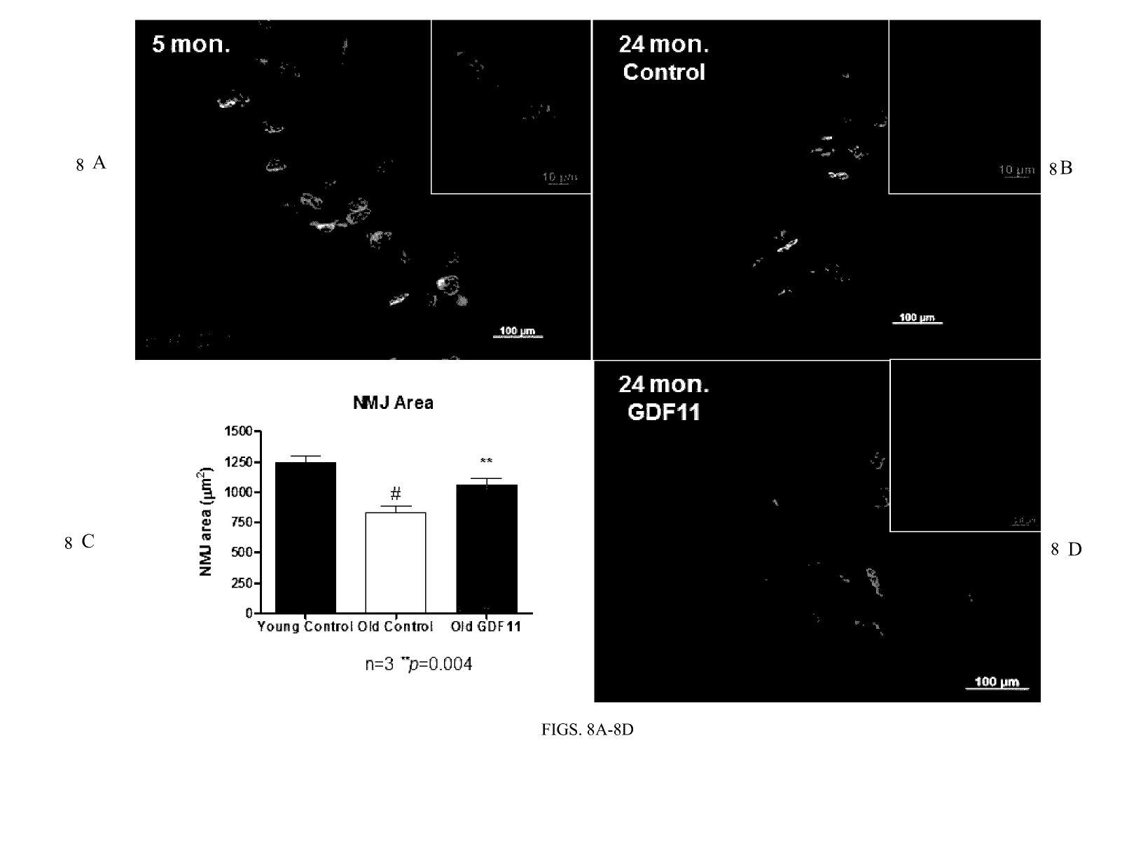

FIG. 9 depicts an alignment of human GDF11 precursor peptide (query sequence; residues 62-407 of SEQ ID NO: 1) and human GDF8 precursor peptide.

FIG. 10 depicts an alignment of human GDF11 precursor peptide (query sequence: residues 47-407 of SEQ ID NO: 1) and murine GDF11 precursor peptide.

FIG. 11 shows the amino acid sequence encoding a human GDF11 precursor polypeptide (SEQ ID NO: 1).

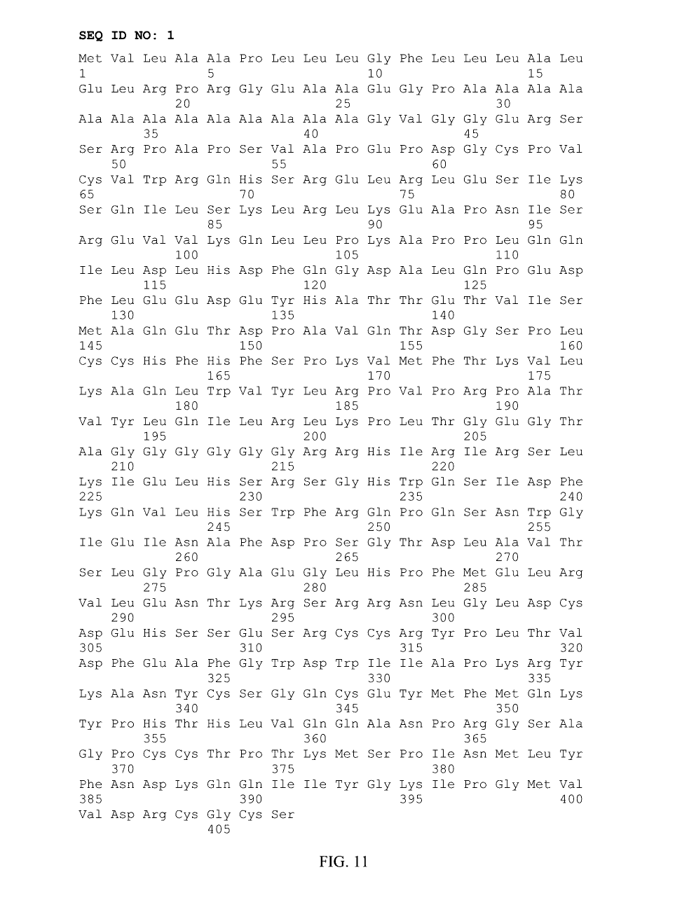

FIG. 12 shows the amino acid sequence encoding a human GDF11 pro-peptide (SEQ ID NO: 2).

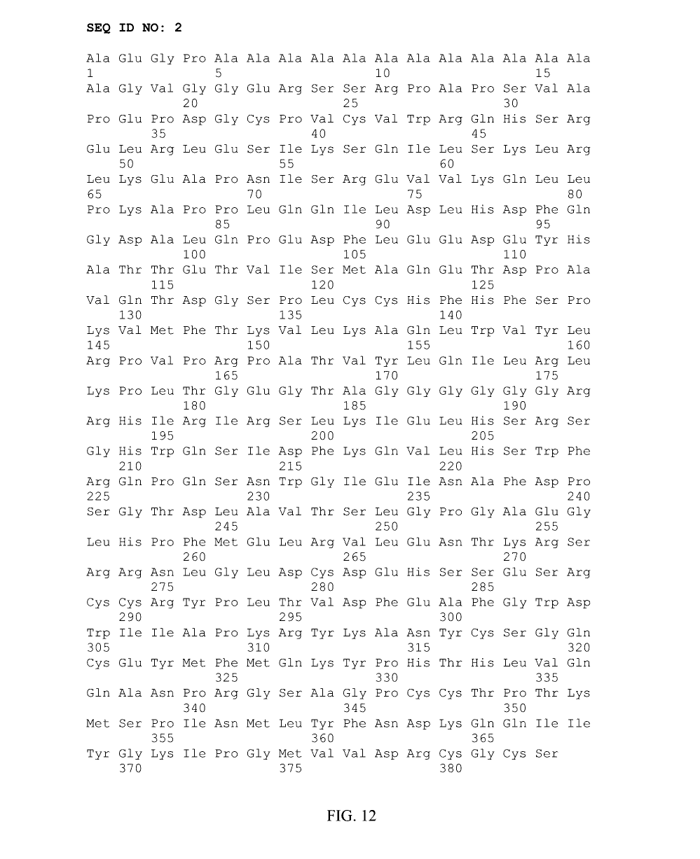

FIG. 13 shows the amino acid sequence encoding a human mature GDF11 polypeptide (SEQ ID NO: 3).

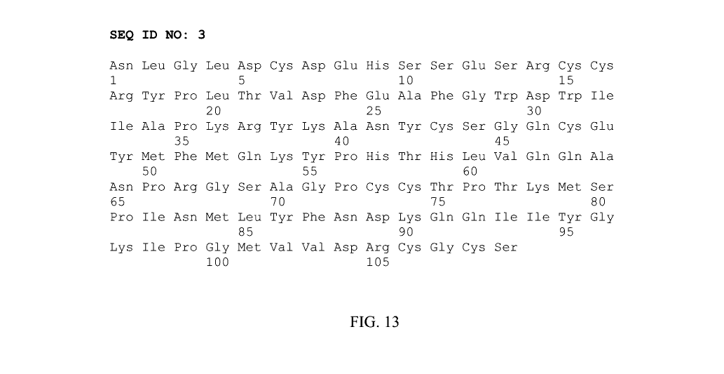

FIG. 14 shows the amino acid sequence encoding a human GDF11 N-terminal polypeptide (SEQ ID NO: 4).

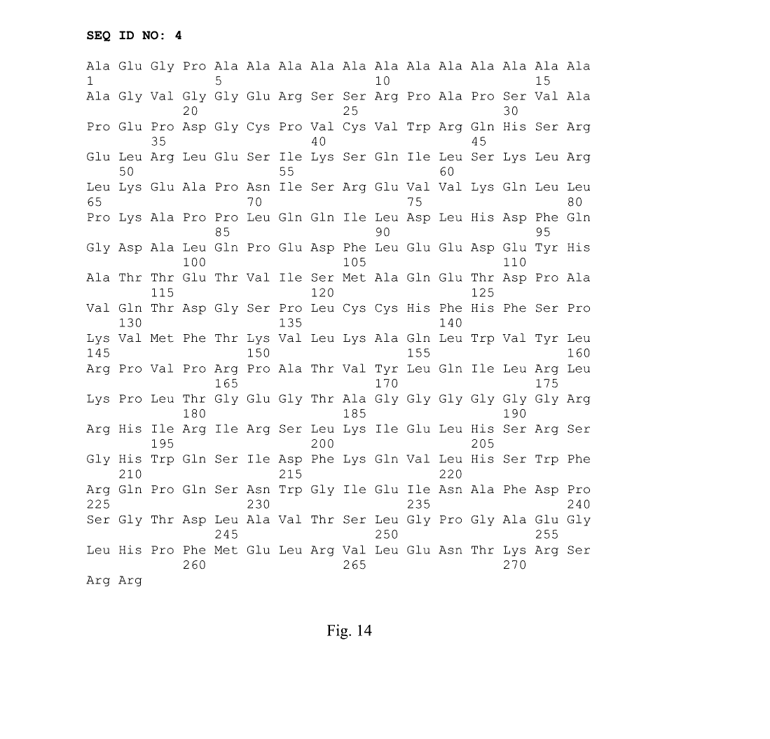

### DETAILED DESCRIPTION OF THE INVENTION

Described herein are methods and compositions based on the discovery that as animals age, the level of GDF11 polypeptide in their blood decreases and results in diminished neuromuscular junction regenerative potential due in part to age-associated degeneration of the neuromuscular junction (e.g., neuromuscular junction fragmentation). The methods and compositions described herein are useful for rejuvenating neuromuscular junctions, treating, preventing, or delaying the onset of neuromuscular junction fragmentation and related disorders, neuromuscular junction degeneration and related disorders, skeletal muscle conditions (e.g., muscle atrophy), motor neuron degeneration and related disorders, and neuromuscular diseases (e.g., amyotrophic lateral sclerosis (ALS)). The methods and compositions described herein generally relate to increasing the level of GDF11 polypeptide or responsiveness to GDF11 polypeptide in a subject to treat, prevent, delay the onset of, or reverse the conditions, diseases, and disorders described herein.

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein, “rejuvenating neuromuscular junctions” refers to at least partially reversing one or more age-related changes in neuromuscular junctions. Non-limiting examples of age-related changes to neuromuscular junctions include: neuromuscular junction fragmentation, reduced or impaired neuromuscular innervation, neuromuscular denervation, loss of motor units, reduced postsynaptic endplate length, decreased density of postsynaptic folds, pathologic neuromuscular junction morphology, increased denervation of fast-twitch muscle fibers, reduced number of nerve terminal branches per endplate, reduced number of terminal sprouts, reduced number of synaptic vesicles in presynaptic terminals, reduced mitochondrial content in presynaptic terminals, reduced nerve terminal area in presynaptic terminals, aberrant quantal content of neurotransmitter release measured by increases in amplitude of evoked endplate potentials (EPP), reduced axonal transport, atrophied or necrotic muscle fibers, reduced acetylcholine receptor expression or number, reduced number of functional motor units in fast-twitch muscle fibers, impaired neuromuscular recovery, diminished physical performance, and reduced muscle size. In some embodiments, at least one age-related neuromuscular junction change is at least partially reversed. In some embodiments, at least two age-related neuromuscular junction changes are at least partially reversed. In some embodiments, at least three age-related neuromuscular junction changes are at least partially reversed. In some embodiments, at least four age-related neuromuscular junction changes are at least partially reversed. In some embodiments, the age-related neuromuscular junction changes are partially reversed by at least 1%, at least 2% at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 33%, at least 35%, at least 41%, at least 44%, or at least 50%. In some embodiments, the age related neuromuscular junction changes are reversed by at least 60%, at least 65%, at least 70%, at least 75%, at least 80%, or at least 90%. In some embodiments, the age-related neuromuscular junction changes are almost completely reversed. In some embodiments, the age-related neuromuscular junction changes are completely reversed.

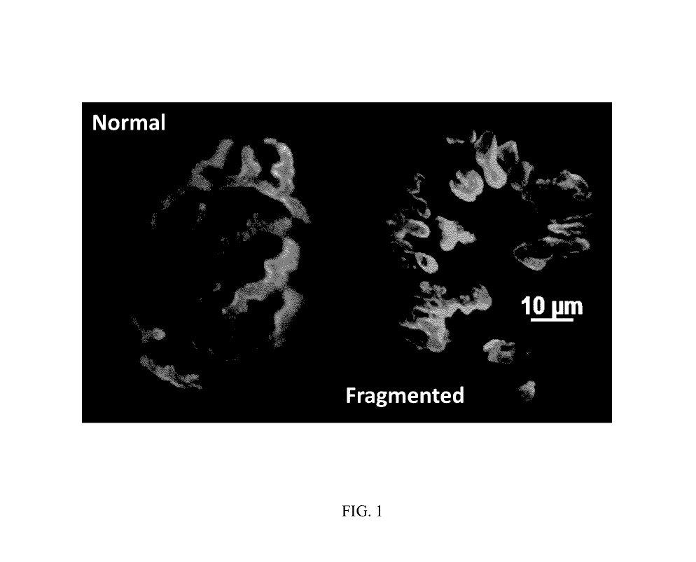

As used herein, “neuromuscular junction fragmentation,” “fragmented neuromuscular junction,” and similar phraseology, refers a neuromuscular junction exhibiting four or more islands, for example, as is shown in FIG. 1. In some contexts, “neuromuscular junction fragmentation” refers to neuromuscular junction fragmentation mediated or characterized by a reduction in circulating GDF11 polypeptide in a subject. As used herein in connection with “neuromuscular junction fragmentation,” a “related disorder” refers to any disorder which involves neuromuscular junction fragmentation. One non-limiting example of such a related disorder is amyotrophic lateral sclerosis (see, e.g., Krakora et al., “Neuromuscular Junction Protection for the Potential Treatment of Amyotrophic Lateral Sclerosis,” Neurology Research International 2012, Article ID 379657, available on the worldwide web). Other examples of disorders related to neuromuscular junction fragmentation are apparent to the skilled artisan.

As used herein, “neuromuscular junction degeneration” refers to the deterioration of any portion (e.g., structural, electrical, mechanical, biochemical, genetic, etc.) of the junction between a nerve fiber and the muscle innervated by the nerve fiber. As used herein in connection with “neuromuscular junction degeneration,” a “related disorder” refers to any disorder which involves neuromuscular junction degeneration. In some contexts, “neuromuscular junction degeneration” refers to neuromuscular junction degeneration mediated or characterized by a reduction in circulating GDF11 polypeptide in a subject.

As used herein, “skeletal muscle condition” refers to a condition in skeletal muscle mediated or characterized by a reduction in circulating GDF11 polypeptide in a subject. Non-limiting examples of skeletal muscle conditions include atrophy, bony fractures associated with muscle wasting or weakness, cachexia, denervation, diabetes, dystrophy, exercise-induced skeletal muscle fatigue, fatigue, frailty, inflammatory myositis, metabolic syndrome, neuromuscular disease, obesity, post-surgical muscle weakness, post-traumatic muscle weakness, sarcopenia, toxin exposure, wasting, and weakness.

As used herein, “frailty” is a syndrome characterized by meeting at least one of the following five attributes: unintentional weight loss, muscle weakness, slow walking speed, exhaustion, and low physical activity.

As used herein, “cachexia” means a state often associated with cancer or other serious diseases or conditions, (e.g., chronic obstructive pulmonary disease, chronic kidney disease), that is characterized by progressive weight loss, muscle atrophy and fatigue, due to the deletion of adipose tissue and skeletal muscle.

As used herein, “post-surgical muscle weakness” refers to a reduction in the strength of one or more muscles following surgical procedure. Weakness may be generalized (i.e., total body weakness) or localized to a specific area, side of the body, limb, or muscle.

As used herein, “post-traumatic muscle weakness” refers to a reduction in the strength of one or more muscles following a traumatic episode (e.g., bodily injury). Weakness may be generalized (i.e., total body weakness) or localized to a specific area, side of the body, limb, or muscle.

As used herein, the phrase “motor neuron degeneration” or “degeneration of motor neuron” means a condition of deterioration of motor neurons, wherein the neurons die or change to a lower or less functionally-active form. Motor neuron degeneration is a hallmark of motor neuron diseases. The motor neuron diseases (MND) are a group of neurodegenerative disorders that selectively affect motor neurons, the nerve cells that control voluntary muscle activity including speaking, walking, breathing, swallowing and general movement of the body. Skeletal muscles are innervated by a group of neurons (lower motor neurons) located in the ventral horns of the spinal cord which project out the ventral roots to the muscle cells. These nerve cells are themselves innervated by the corticospinal tract or upper motor neurons that project from the motor cortex of the brain. On macroscopic pathology, there is a degeneration of the ventral horns of the spinal cord, as well as atrophy of the ventral roots. In the brain, atrophy may be present in the frontal and temporal lobes. On microscopic examination, neurons may show spongiosis, the presence of astrocytes, and a number of inclusions including characteristic “skein-like” inclusions, bunina bodies, and vacuolisation. Motor neuron diseases are varied and destructive in their effect. They commonly have distinctive differences in their origin and causation, but a similar result in their outcome for the patient: severe muscle weakness. Amyotrophic lateral sclerosis (ALS), primary lateral sclerosis (PLS), progressive muscular atrophy (PMA), pseudobulbar palsy, progressive bulbar palsy, spinal muscular atrophy (SMA) and post-polio syndrome are all examples of MND. The major site of motor neuron degeneration classifies the disorders.

Common MNDs include amyotrophic lateral sclerosis, which affects both upper and lower motor neurons. Progressive bulbar palsy affects the lower motor neurons of the brain stem, causing slurred speech and difficulty chewing and swallowing. Individuals with these disorders almost always exhibit abnormal signs in the arms and legs. Primary lateral sclerosis is a disease of the upper motor neurons, while progressive muscular atrophy affects only lower motor neurons in the spinal cord. Means for diagnosing MND are well known to those skilled in the art. Non-limiting examples of symptoms, utilizing ALS as an example, are described below.

Amyotrophic lateral sclerosis (ALS), also called Lou Gehrig's disease or classical motor neuron disease, is a progressive, ultimately fatal disorder that eventually disrupts signals to all voluntary muscles. In the United States, doctors use the terms motor neuron disease and ALS interchangeably. Both upper and lower motor neurons are affected. Approximately 75 percent of people with classic ALS will also develop weakness and wasting of the bulbar muscles (muscles that control speech, swallowing, and chewing). Symptoms are usually noticed first in the arms and hands, legs, or swallowing muscles. Muscle weakness and atrophy occur disproportionately on both sides of the body. Affected individuals lose strength and the ability to move their arms, legs, and body. Other symptoms include spasticity, exaggerated reflexes, muscle cramps, fasciculations, and increased problems with swallowing and forming words. Speech can become slurred or nasal. When muscles of the diaphragm and chest wall fail to function properly, individuals lose the ability to breathe without mechanical support. Although the disease does not usually impair a person's mind or personality, several recent studies suggest that some people with ALS may have alterations in cognitive functions such as problems with decision-making and memory. ALS most commonly strikes people between 40 and 60 years of age, but younger and older people also can develop the disease. Men are affected more often than women. Most cases of ALS occur sporadically, and family members of those individuals are not considered to be at increased risk for developing the disease. However, there is a familial form of ALS in adults, which often results from mutation of the superoxide dismutase gene, or SOD1, located on chromosome 21. In addition, a rare juvenile-onset form of ALS is genetic. Most individuals with ALS die from respiratory failure, usually within 3 to 5 years from the onset of symptoms. However, about 10 percent of affected individuals survive for 10 or more years.

As used herein, “neuromuscular disease” means any disease or condition that affects any part of the nerve and muscle. Neuromuscular disease encompasses critical illness polyneuropathy, prolonged neuromuscular blockade, acute myopathy as well as acute inflammatory demyelinating polyradiculoneuropathy, amyotrophic lateral sclerosis (ALS), autonomic neuropathy, Charcot-Marie-Tooth disease and other hereditary motor and sensory neuropathies, chronic inflammatory demyelinating polyradiculoneuropathy, dermatomyositis/polymyositis, diabetic neuropathy, dystrophinopathies, endocrine myopathies, focal muscular atrophies, hemifacial spasm, hereditary neuropathies of the Charcot-Marie-Tooth disease type, inclusion body myositis, Kennedy disease, Lambert-Eaton myasthenic syndrome, muscular dystrophy (e.g., limb-girdle, Duchenne, Becker, myotonic, facioscapulohumeral, etc.), metabolic myopathies, metabolic neuropathy, multifocal motor neuropathy with conduction blocks, myasthenia gravis, neuropathy of Friedreich Ataxia, neuropathy of leprosy, nutritional neuropathy, periodic paralyses, primary lateral sclerosis, restrictive lung disease, sarcoidosis and neuropathy, Schwartz-Jampel Syndrome, spinal muscular atrophy (SMA), stiff person syndrome, thyroid disease, traumatic peripheral nerve lesions, vasculitic neuropathy, among others.

As used herein, “sarcopenia” means a loss of skeletal muscle mass, quality, and strength. Often sarcopenia is associated with aging, but may also occur in association with HIV infection and a variety of chronic conditions. Sarcopenia may lead to frailty, for example, in the elderly. Sarcopenia also encompasses a condition or symptom associated with sarcopenia including, but not limited to loss of skeletal muscle mass, muscle weakness, fatigue, disability, and morbidity.

Those skilled in the art will appreciate that certain of the conditions, diseases, and disorders described herein may appropriately reside in multiple categories of conditions, diseases, and disorders in accordance with the present invention. For example, amyotrophic lateral sclerosis is considered to be a motor neuron disease and a neuromuscular disease in accordance with the present invention, as well as a disorder related to neuromuscular junction fragmentation, as is evidenced by role of neuromuscular junction fragmentation in amyotrophic lateral sclerosis (see, e.g., Krakora et al., 2008).

The terms “decrease,” “reduce,” “reduced,” “reduction,” “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased,” “increase” or “enhance” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more as compared to a reference level.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.”

The term “biological sample” as used herein denotes a sample taken or isolated from a biological organism, e.g., skeletal muscle sample, blood sample, cell lysate, a homogenate of a tissue sample from a subject, or a fluid sample from a subject. Exemplary biological samples include, but are not limited to, skeletal muscle tissue biopsies or blood and/or serum samples. In some embodiments, the sample is from a resection, biopsy, or core needle biopsy. In addition, fine needle aspirate samples can be used. Samples can include paraffin-embedded and frozen tissue. The term “biological sample” also includes untreated or pretreated (or pre-processed) biological samples. In some embodiments, the biological sample is an untreated biological sample. The sample can be obtained by removing a sample of cells from a subject, but can also be accomplished by using previously isolated cells (e.g. isolated at a prior time point and isolated by the same or another person).

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient”, “individual” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, of neuromuscular junction deterioration, degeneration, fragmentation, and related disorders, motor neuron degeneration and related disorders, skeletal muscle conditions (e.g., muscle atrophy), or neuromuscular disease (e.g., amyotrophic lateral sclerosis). In addition, the methods described herein can be used to treat domesticated animals and/or pets. A subject can be male or female. A subject can be one who has been previously diagnosed with or identified as suffering from or having a condition, disease, or disorder described herein in need of treatment (e.g., of neuromuscular junction deterioration, degeneration, fragmentation, and related disorders, motor neuron degeneration and related disorders, skeletal muscle conditions (e.g., muscle atrophy), or neuromuscular disease (e.g., amyotrophic lateral sclerosis)) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors for a condition or one or more complications related to a condition. A “subject in need” of treatment for a particular condition can be a subject having that condition, diagnosed as having that condition, or at increased risk of developing that condition relative to a given reference population.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The term “statistically significant” or “significantly” refers to statistical significance and generally means a p value greater than 0.05 (calculated by the relevant statistical test). Those skilled in the art will readily appreciate that the relevant statistical test for any particular experiment depends on the type of data being analyzed. Additional definitions are provided in the text of individual sections below.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001) and Davis et al., Basic Methods in Molecular Biology, Elsevier Science Publishing, Inc., New York, USA (1995) which are both incorporated by reference herein in their entireties.

Described herein are methods comprising administering to a subject a composition which increases the level or concentration of GDF11 polypeptide in the subject. In some embodiments, the subject is one who has, or has been diagnosed as having a condition, disease, or disorder described herein due to aging. As is used herein, a condition, disease, or disorder described herein “due to aging” refers to one which is at least partially attributable to a subject's age.

In some embodiments, the subject is one who has, or has been diagnosed as having neuromuscular junction fragmentation, for example due to aging, or a related disorder. In some embodiments, the subject is one who is at risk of developing a disorder associated with neuromuscular junction fragmentation, for example, due to aging. In some embodiments, the subject is one who exhibits neuromuscular junction fragmentation. In some embodiments, the subject is one who exhibits one or more symptoms of a disorder involving neuromuscular junction fragmentation. In some embodiments, the subject is one who exhibits one or more age-related symptoms of a disorder involving neuromuscular junction fragmentation.

In some embodiments, the subject is one who has, or has been diagnosed as having neuromuscular junction degeneration, for example due to aging, or a related disorder. In some embodiments, the subject is one who is at risk of developing a disorder associated with neuromuscular junction degeneration, for example, due to aging. In some embodiments, the subject is one who exhibits neuromuscular junction degeneration. In some embodiments, the subject is one who exhibits one or more symptoms of a disorder involving neuromuscular junction degeneration. In some embodiments, the subject is one who exhibits one or more age-related symptoms of a disorder involving neuromuscular junction degeneration.

In some embodiments, the subject is one who has, or has been diagnosed as having motor neuron degeneration, for example due to aging, or a related disorder. In some embodiments, the subject is one who is at risk of developing a disorder associated with motor neuron degeneration, for example, due to aging. In some embodiments, the subject is one who exhibits motor neuron degeneration. In some embodiments, the subject is one who exhibits one or more symptoms of a disorder involving motor neuron degeneration. In some embodiments, the subject is one who exhibits one or more age-related symptoms of a disorder involving motor neuron degeneration.

In some embodiments, the subject is one who has, or has been diagnosed as having a skeletal muscle condition (e.g., muscle atrophy), for example due to aging, or a related disorder. In some embodiments, the subject is one who is at risk of developing a skeletal muscle condition, for example, due to aging. In some embodiments, the subject is one who exhibits one or more symptoms of a skeletal muscle condition. In some embodiments, the subject is one who exhibits one or more age-related symptoms of a skeletal muscle condition.

In some embodiments, the subject is one who has, or has been diagnosed as having a neuromuscular disease (e.g., amyotrophic lateral sclerosis), for example, due to aging. In some embodiments, the subject is one who is at risk of developing a neuromuscular disease, for example, due to aging. In some embodiments, the subject is one who exhibits one or more symptoms of a neuromuscular disease. In some embodiments, the subject is one who exhibits one or more age-related symptoms of a neuromuscular disease.

In some embodiments, the subject is an elderly subject. In some embodiments, an elderly subject is over the age of 50, 55, 60, 65, 70, 75, 80, 85, 90, or 100 years.

In some embodiments, the composition which increases the level of GDF11 polypeptide is administered to a subject who has or has been diagnosed with a neuromuscular disease described herein.

In some embodiments, the level of GDF11 polypeptide is the level of GDF11 in the circulation of a subject. In some embodiments, the level of GDF11 polypeptide is the level of GDF11 in the skeletal muscle tissue of a subject. In some embodiments, the level of GDF11 polypeptide is determined by measuring the level of an mRNA encoding a GDF11 polypeptide. The level of GDF11 in a subject can be determined by obtaining a biological sample from the subject and determining the level of GDF11 in the biological sample. Methods for determining the level of a polypeptide in a subject or a sample obtained from a subject are well known in the art and include, but are not limited to, ELISA, radioimmunoassay, immunohistochemistry, methods involving a labeled antibody specific for GDF11, dot blot analysis, functional bioassays, Northern blot, in-situ hybridization, and RT-PCR, aptamer-based proteomic technology (e.g., SOMAscan™ commercially available from SomaLogic, Inc.) among others. Antibodies specific for GDF11 are commercially available, e.g. Cat. No. ab71347 from Abcam: Cambridge, Mass. In some embodiments, the antibodies are antibodies which do not cross-react with GDF8. In some embodiments, the antibodies are selective GDF11 monoclonal antibodies. In some embodiments, the level of GDF11 can be measured as described in Souza et al., Molecular Endocrinology 2008 22:2689-2702; which is incorporated by reference herein in its entirety.

As animals age, neuromuscular junctions often degenerate and experience diminished regenerative potential due to deterioration of neuromuscular junctions (e.g., neuromuscular junction fragmentation), which may be associated with one or more skeletal muscle conditions (e.g., muscle atrophy) and/or one or more neuromuscular diseases (e.g., amyotrophic lateral sclerosis). Without wishing to be bound by theory, it is believed that neuromuscular junction degeneration, fragmentation, and deterioration, and characteristic attendant reduced skeletal muscle size and pathologic morphology results in part from decreased levels of circulating GDF11 polypeptide. The work described herein demonstrates that decreased levels of circulating GDF11 polypeptide results in diminished neuromuscular junction regenerative potential due in part to fragmentation of neuromuscular junctions. The work described herein surprisingly and unexpectedly demonstrates that GDF11 polypeptide rejuvenates neuromuscular junctions, and actually reduces or reverses neuromuscular junction fragmentation, thereby improving the regenerative potential of neuromuscular junctions, improving neuromuscular junction and skeletal muscle morphology and ultrastructure, improving physical performance, and improving neuromuscular recovery.

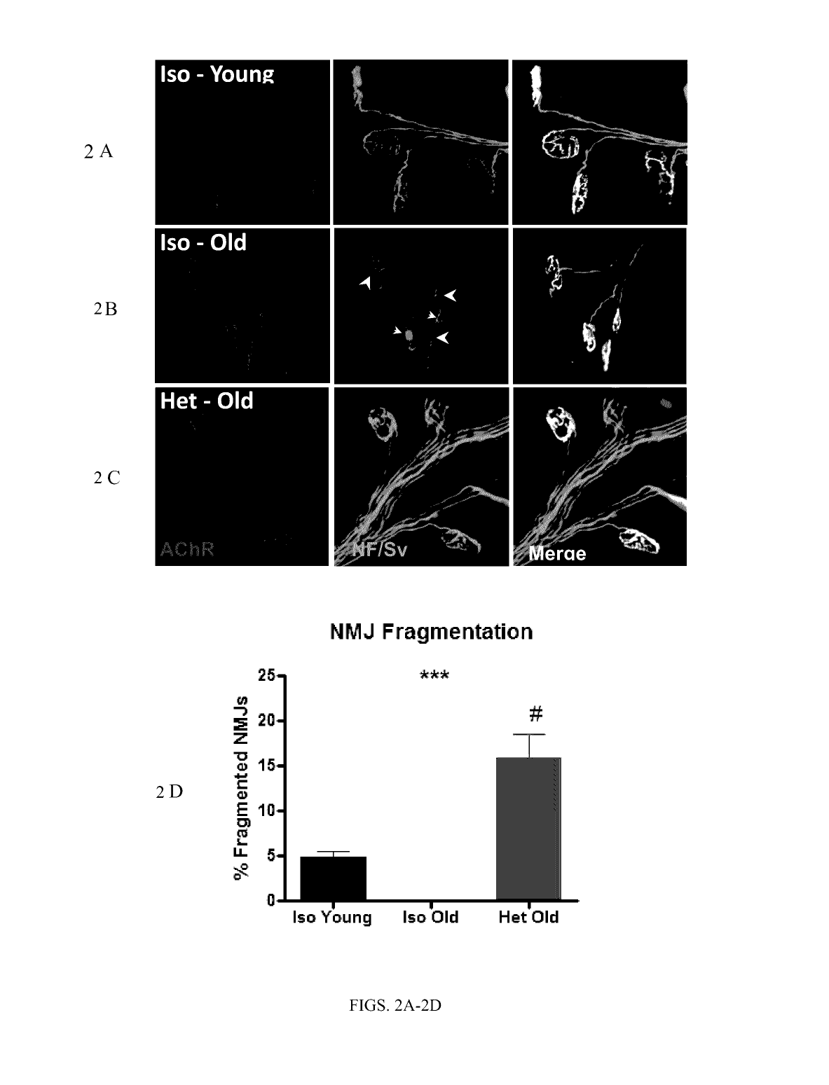

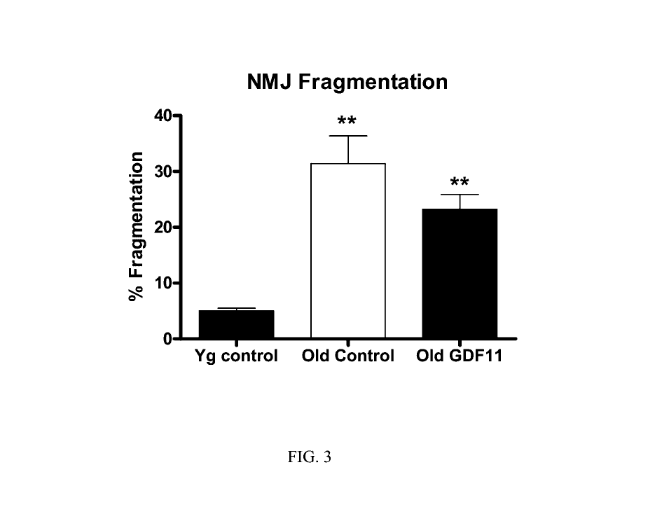

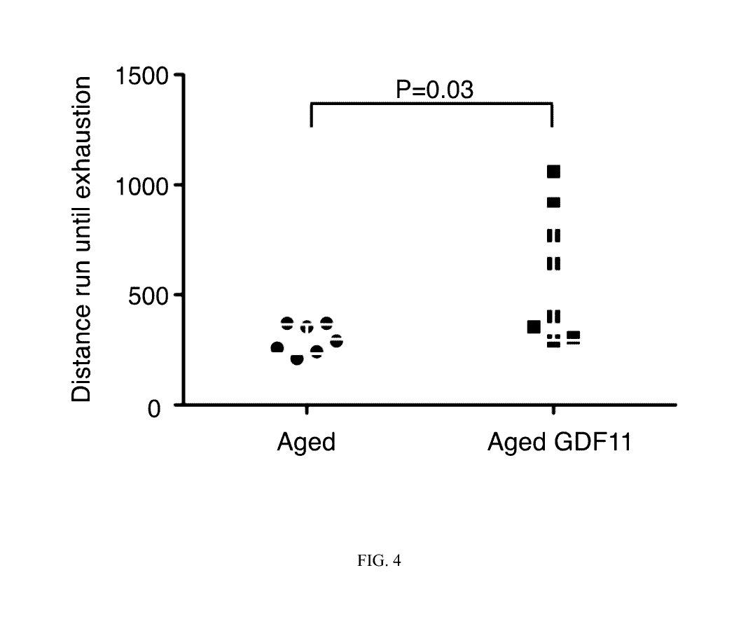

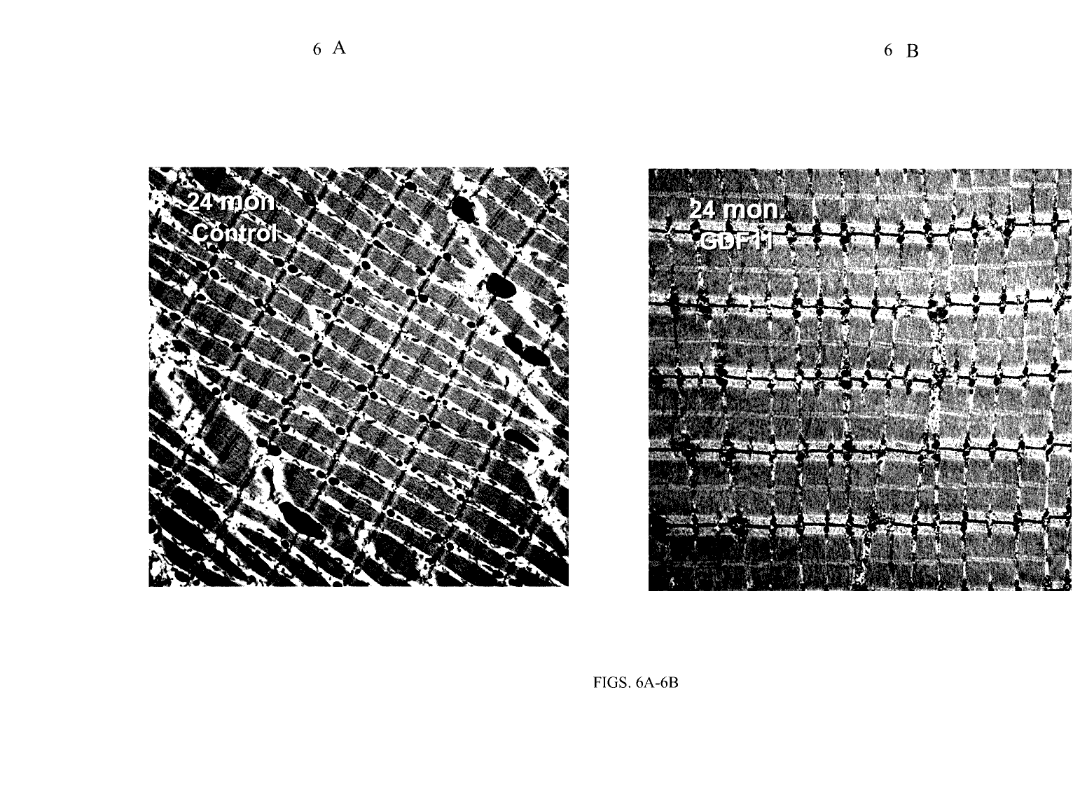

Surprisingly, the work described herein demonstrates that aged mice treated in vivo with daily IP injection of recombinant GDF11 (rGDF11) reduced fragmentation in the subject's neuromuscular junctions (FIGS. 2D and 3), enhanced the subject's physical performance (FIG. 4), and increased the subject's muscle size (FIG. 6D).

Accordingly, in one aspect, the present invention provides a method of rejuvenating neuromuscular junctions in a subject in need thereof, the method comprising administering to the subject a composition which increases the level of GDF11 polypeptide in the subject. In some embodiments, increased levels of GDF11 polypeptide in the subject rejuvenate the subject's neuromuscular junctions.

In another aspect, the present invention provides a method of treating, preventing, or delaying the onset of, neuromuscular junction fragmentation or a related disorder in a subject in need thereof, the method comprising administering to the subject a composition which increases the level of GDF11 polypeptide in the subject. In some embodiments, increased levels of GDF11 polypeptide in the subject reverses neuromuscular junction fragmentation, thereby treating, preventing, or delaying the onset of, a disorder related to neuromuscular junction fragmentation. In one embodiment, a disorder related to neuromuscular junction fragmentation comprises amyotrophic lateral sclerosis.

In another aspect, the present invention provides a method of treating, preventing, or delaying the onset of, neuromuscular junction degeneration or a related disorder in a subject in need thereof, the method comprising administering to the subject an effective amount of a composition which increases the level of GDF11 polypeptide in the subject. In some embodiments, increased levels of GDF11 polypeptide in the subject reverses neuromuscular junction fragmentation and/or reverses pathologic neuromuscular junction morphology, and/or improves muscle ultrastructure, thereby treating, preventing, or delaying the onset of, neuromuscular junction degeneration or a disorder related to neuromuscular junction degeneration in the subject. In one embodiment, a disorder related to neuromuscular junction degeneration comprises amyotrophic lateral sclerosis.

In another aspect, the present invention provides a method of treating, preventing, or delaying the onset of, motor neuron degeneration in a subject in need thereof, the method comprising administering to the subject an effective amount of a composition which increases the level of GDF11 polypeptide in the subject.

In another aspect, the present invention provides a method of treating, preventing, or delaying the onset of, muscle atrophy in a subject in need thereof, the method comprising administering to the subject an effective amount of a composition which increases the level of GDF11 polypeptide in the subject.

In another aspect, the present invention provides a method of treating, preventing, or delaying the onset of, a neuromuscular disease in a subject in need thereof, the method comprising administering to the subject an effective amount of a composition which increases the level of GDF11 polypeptide in the subject. In one embodiment, the neuromuscular disease comprises amyotrophic lateral sclerosis.

In another aspect, the present invention provides a method of treating, preventing, or delaying the onset of amyotrophic lateral sclerosis in a subject in need thereof the method comprising administering to the subject an effective amount of a composition which increases the level of GDF11 polypeptide in the subject.

The methods and compositions described herein relate to increasing the level of GDF11 polypeptide in a subject. As used herein, “GDF11” refers to “Growth and Differentiation Factor 11” (NCBI Gene ID No: 10220), a member of the Transforming Growth Factor-beta superfamily of growth factors. GDF11 is known to bind TGFβ3 superfamily type I receptors including ALK4, ALK5, and ALK7. For signaling in mammalian development, GDF11 predominantly uses ALK4 and ALK5. In some embodiments, GDF11 signaling can also occur via the ACVR2B receptor. GDF11 is also closely related to GDF8 (also known as myostatin). GDF11 can also be referred to as bone morphogenic protein 11, i.e. BMP11. As used herein, “GDF11” can include the human precursor polypeptide (SEQ ID NO: 1, NCBI Ref Seq: NP 005802); the human pro-peptide (SEQ ID NO: 2); the human N-terminal polypeptide (SEQ ID NO: 4), and the human mature (SEQ ID NO: 3) forms of GDF11 as well as homologs from other species, including but not limited to bovine, dog, cat, chicken, murine, rat, porcine, bovine, turkey, horse, fish, baboon and other primates. The terms also refer to fragments or variants of GDF11 that maintain at least 50% of the neuromuscular junction rejuvenating effect of the full length GDF11 of SEQ ID NO: 2, SEQ ID NO: 1, or SEQ ID NO: 3, e.g. as measured in an appropriate animal model (e.g., heterochronic parabiosis of aged mice).

Conservative substitution variants that maintain the neuromuscular junction rejuvenating effect of wild type GDF11 will include a conservative substitution as defined herein. The identification of amino acids most likely to be tolerant of conservative substitution while maintaining at least 50% of the activity of the wild type GDF11 is guided by, for example, sequence alignment with GDF11 homologs or paralogs from other species. Amino acids that are identical between GDF11 homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants can be tested for activity, for example, by administering the variant to an appropriate animal model (e.g., cryoinjured aged mice to induce post-injury regeneration).

For human GDF11, the pro-peptide plus signal sequence (e.g. the precursor polypeptide) is 407 amino acids long. Cleavage of the 24 amino acid signal peptide generates a pro-peptide of 383 amino acids and cleavage of the pro-peptide results in a mature GDF11 polypeptide of 109 amino acids that corresponds to the C-terminal 109 amino acids of the pro-peptide. The mature polypeptide forms a disulfide-linked homodimer. Cleavage of the pro-peptide also generates the N-terminal polypeptide (e.g., SEQ ID NO: 4) comprising amino acids 25-298 of SEQ ID NO: 1. The N-terminal GDF11 polypeptide can antagonize the activity of, e.g., the polypeptides of SEQ ID NOs: 2 and 3, at least in vitro by forming a complex with other forms of GDF11 polypeptides and can thus be used to modulate the activity of GDF11 compositions as described herein. Thus, to the extent that GDF11 polypeptides as described herein rejuvenate neuromuscular junctions or promote neuromuscular junction regeneration, and to the extent the N-terminal GDF11 polypeptide of, e.g., SEQ ID NO: 4, can antagonize such effects, the polypeptide of SEQ ID NO: 4 can be excluded from the meaning of “GDF11 polypeptide” as that term is used herein.

As used herein, the terms “proteins” and “polypeptides” are used interchangeably to designate a series of amino acid residues connected to the other by peptide bonds between the alpha-amino and carboxy groups of adjacent residues. The terms “protein,” and “polypeptide” refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when refining to a gene product and fragments thereof.

Thus, exemplary polypeptides or proteins include gene products, naturally occurring proteins, homologs, orthologs, paralogs, fragments and other equivalents, variants, fragments, and analogs of the foregoing.

As used herein, “pro-peptide” used in reference to GDF11 refers to a GDF11 polypeptide in which the signal domain (e.g. amino acids 1-24 of SEQ ID NO: 1) has been cleaved off during formation of the mature and/or active forms of GDF11. As used herein, “precursor peptide” used in reference to a GDF11 polypeptide comprising the signal domain, e.g., a polypeptide comprising the amino acid sequence of SEQ ID NO: 1.

In some embodiments, the level of GDF11 in a subject is increased by administering a composition comprising a GDF11 agonist that increases the level or activity of GDF11 in the subject. Exemplary GDF11 agonists are described in U.S. Pat. No. 8,323,964.

In some embodiments, the level of GDF11 in a subject is increased by administering a composition comprising an agonist antibody that increases expression of GDF11 in the subject. In some embodiments, the level of GDF11 in a subject is increased by administering a composition comprising an agonist antibody that increases activity of GDF11 in the subject. Any antibody that is capable of increasing expression or activity of GDF11 in the subject can be used. As used herein “antibody” includes, but is not limited to, full length antibodies, antibody fragments, single chain antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Exemplary antibody fragments include, but are not limited to, (i) the Fab fragment consisting of VL, VH, CL and CH I domains, (ii) the Fd fragment consisting of the VH and CHI domains, (iii) the Fv fragment consisting of the VL and VH domains of a single antibody, (iv) the dAb fragment, which consists of a single variable domain, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site, (viii) bispecific single chain Fv dimers, and (ix) “diabodies” or “triabodies,” multivalent or multispecific fragments constructed by gene fusion. The antibody fragments may be modified. For example, the molecules may be stabilized by the incorporation of disulfide bridges linking the VH and VL domains. Examples of antibody formats and architectures are described in Holliger & Hudson, 2006, Nature Biotechnology 23(9): 1 126-1 136, and Carter 2006, Nature Reviews Immunology 6:343-357 and references cited therein, all expressly incorporated by reference. Exemplary GDF11 agonist antibodies include the GDF11 agonist antibodies described in PCT International Application Publication WO/2002/010214.

In some embodiments, the level of GDF11 in a subject is increased by administering a composition comprising a GDF11 polypeptide and/or a nucleic acid encoding a GDF11 polypeptide. A GDF11 polypeptide administered to a subject according to the methods described herein can comprise a GDF11 polypeptide as described herein above, e.g. a pro-peptide or mature form. In some embodiments, the GDF11 polypeptide comprises the amino acid sequence of SEQ ID NO: 1. In some embodiments, the GDF11 polypeptide comprises the amino acid sequence of SEQ ID NO: 2. In some embodiments, the GDF11 polypeptide comprises the amino acid sequence of SEQ ID NO: 3. In some embodiments, the GDF11 polypeptide comprises the amino acid sequence of SEQ ID NO: 4.

In some embodiments, the composition administered to the subject can comprise GDF11 polypeptide homodimers comprising polypeptides of the amino acid sequence of SEQ ID NO: 3. In some embodiments, the composition administered to the subject can comprise GDF11 polypeptide homodimers comprising polypeptides of the amino acid sequence of SEQ ID NO: 4. In some embodiments, the composition administered to the subject can comprise GDF11 polypeptide homodimers comprising polypeptides of the amino acid sequence of SEQ ID NO: 2. In some embodiments, the composition administered to the subject can comprise GDF11 polypeptide homodimers comprising polypeptides of the amino acid sequence of SEQ ID NO: 1. In some embodiments, the composition administered to the subject can comprise GDF11 polypeptide heterodimers comprising polypeptides of any of the amino acid sequence of SEQ ID NO: 4, SEQ ID NO: 3, SEQ ID NO: 2, and/or SEQ ID NO: 1.

In some embodiments, a variant or fragment of a GDF11 polypeptide can be administered to a subject. In some embodiments, the variant of GDF11 is a conservatively modified variant. In some embodiments of any of the aspects described herein, the subject can be administered a variant or fragment (e.g. a conservatively modified variant or a functional fragment or a nucleic acid encoding such a polypeptide) of a polypeptide selected from Collectin kidney 1 (e.g. NCBI Gene ID No: 78989), Cathespin D (e.g. NCBI Gene ID No: 1509), Dickkopf-related protein 4 (e.g. NCBI Gene ID No: 27121), Erythrocyte membrane protein 4.1 (e.g. NCBI Gene ID No: 2035), esterase D (e.g. NCBI Gene ID No: 2098), hemoglobin (e.g. NCBI Gene ID No: 3043 or 3047), interleukin-1 receptor accessory protein (e.g. NCBI Gene ID No: 3556), natural killer group 2 member D (e.g. NCBI Gene ID No: 22914), Ras-related C3 botulinum toxin substrate 1 (e.g. NCBI Gene ID No: 5879), GTP-binding nuclear protein Ran (e.g. NCBI Gene ID No: 5901), tissue inhibitor of metalloproteases 3 (e.g. NCBI Gene ID No: 7078), and thymidylate synthase (e.g. NCBI Gene ID No: 7298).

In some embodiments, the GDF11 polypeptide can be a variant of a sequence described herein, e.g. a variant of a GDF11 polypeptide comprising the amino acid sequence of SEQ ID NO: 4, SEQ ID NO: 3, SEQ ID NO: 1, or SEQ ID NO: 2. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encodes a variant protein or fragment thereof that retains the relevant biological activity relative to the reference protein, i.e., can rejuvenate neuromuscular junctions at least 50% as well as wild type GDF11. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wild type GDF11, e.g. 110%, 125%, 150%, 175%, 200%, 500%, 1000% or more.

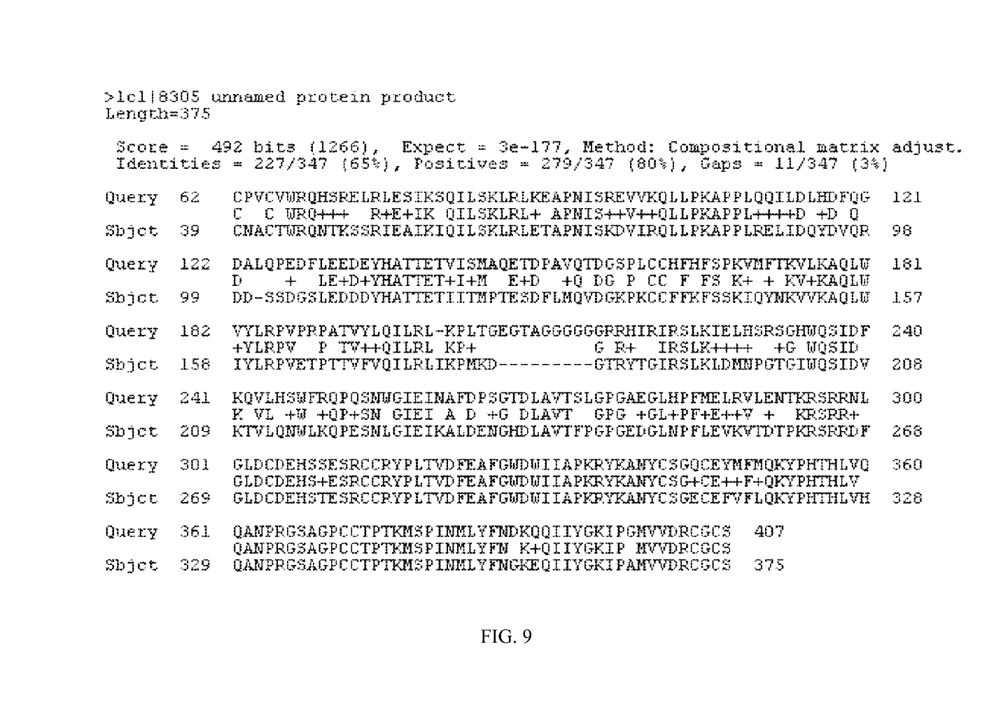

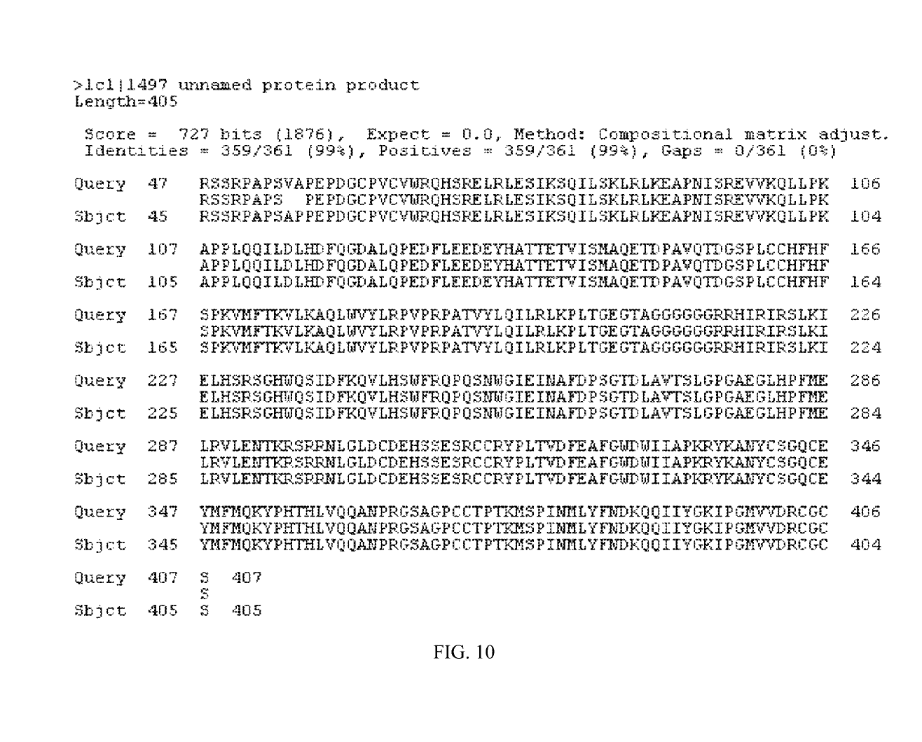

One method of identifying amino acid residues which can be substituted is to align, for example, human GDF11 to a GDF11 homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. Similarly, alignment with a related polypeptide from the same species, e.g. GDF8, which does not show the same activity, can also provide guidance with respect to regions or structures required for GDF11 activity. FIG. 9 depicts an example of an alignment between human GDF11 precursor peptide (query sequence; residues 62-407 of SEQ ID NO: 1) and human GDF8 precursor peptide created using the default settings of the alignment tool of the BLASTP program, freely available on the world wide web at http://blast.ncbi.nlm.nih.gov/. FIG. 10 depicts an example of an alignment between human GDF11 precursor peptide (query sequence; residues 47-407 of SEQ ID NO: 1) and murine GDF11 precursor peptide created using the default settings of the alignment tool of the BLASTP program, freely available on the world wide web at http://blast.ncbi.nlm.nih.gov/. The variant amino acid or DNA sequence can be at least 90%, at least 95%, at least 96%, at least 97%, at least 98%, at least 99%, or more, identical to a native or reference sequence, e.g. SEQ ID NO: 4, SEQ ID NO: 3, SEQ ID NO:1, or SEQ ID NO: 2 or a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set.

It is noted that the mature GDF11 polypeptide includes likely intrachain disulfide bonds between, e.g. amino acid 313 and 372; 341 and 404; and 345 and 406 (numbered relative to the full length polypeptide, including the signal sequence) and that amino acid 371 likely participates in interchain disulfide bonding.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired apoptotic activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, the GDF11 polypeptide administered to a subject can comprise one or more amino acid substitutions or modifications. In some embodiments, the substitutions and/or modifications can prevent or reduce proteolytic degradation and/or prolong half-life of the polypeptide in the subject. In some embodiments, a GDF11 polypeptide can be modified by conjugating or fusing it to other polypeptide or polypeptide domains such as, by way of non-limiting example, transferrin (W006096515A2), albumin (Yell et al., 1992), growth hormone (US2003104578AA); cellulose (Levy and Shoseyov, 2002); and/or Fe fragments (Ashkenazi and Chamow, 1997). The references in theforegoing paragraph are incorporated by reference herein in their entireties.

In some embodiments, a GDF11 polypeptide as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Nonlimiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta (aminoalkyl)-phenylacetic acid, thioamide, tetrazole, boronic ester, olefinic group, and derivatives thereof.

In some embodiments, a GDF11 polypeptide as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, a GDF11 polypeptide as described herein can comprise alternative amino acids. Non-limiting examples of alternative amino acids include, D-anlino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, parabenzoylphenylalanine, para-amino phenylalanine, p-fluorophenylalanine, phenylglycine, propargylglycine, sarcosine, and tert-butylglycine), diaminobutyric acid, 7-hydroxytetrahydroisoquinoline carboxylic acid, naphthylalanine, biphenylalanine, cyclohexylalanine, aminoisobutyric acid, norvaline, norleucine, tert-leucine, tetrahydroisoquinoline carboxylic acid, pipecolic acid, phenyiglycine, homophenylalanine, cyclohexylglycine, dehydroleucine, 2,2-diethylglycine, 1-amino-lcyclopentanecarboxylic acid, 1-amino-1-cyclohexanecarboxylic acid, amino-benzoic acid, aminonaphthoic acid, gamma-aminobutyric acid, difluorophenylalanine, nipecotic acid, alpha-anlino butyric acid, thienyl-alanine, t-butylglycine, trifluorovaline; hexafluoroleucine; fluorinated analogs; azidemodified amino acids; alkyne-modified amino acids; cyano-modified amino acids; and derivatives thereof.

In some embodiments, a GDF11 polypeptide can be modified, e.g. by addition of a moiety to one or more of the amino acids comprising the peptide. In some embodiments, a GDF11 polypeptide as described herein can comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. In some embodiments, a GDF11 polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C terminus, introduction of C-terminal alcohol, aldehyde, ester, and thioester moieties. The half-life of a GDF11 polypeptide can be increased by the addition of moieties, e.g. PEG or albumin.

In some embodiments, the GDF11 polypeptide administered to the subject can be a functional fragment of one of the GDF11 amino acid sequences described herein. As used herein, a “functional fragment” is a fragment or segment of a peptide which can rejuvenate neuromuscular junctions in a subject in accordance with the work described herein. A functional fragment can comprise conservative substitutions of the sequences disclosed herein. In some embodiments, a functional fragment can comprise the 12.5 kDa C-terminus of GDF11. In some embodiments, the 12.5 kDa C-terminus of GDF11 can function as a monomer. In some embodiments, the 12.5 kDa C-terminus of GDF11 can function as a homodimer. In some embodiments, the 12.5 kDa C-tenninus of GDF11 can function as a heterodimer with the GDF11 pro-peptide.

Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. In some embodiments, a GDF11 polypeptide as described herein can be chemically synthesized and mutations can be incorporated as part of the chemical synthesis process.

In some embodiments, a GDF11 polypeptide as described herein can be formulated as a pharmaceutically acceptable prodrug. As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biophamzaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chern. 671-696; Asgharnejad, “Improving Oral Drug Transport,” in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration,” Eur. J. Drug Metab, Pharmacokinet., 15(2): 143-53 (1990); Balimane and Sinko, “Involvement of multiple transporters in the oral absorption of nucleoside analogues,” Adv. Drug Delivel)' Rev., 39(1-3): 183-209 (1999); Browne, “Fosphenytoin (Cerebyx),” Clin. Neuropharmacol. 20(1): 1-12 (1997); Bundgaard, “Bioreversible derivatization of drugs—principle and applicability to improve the therapeutic effects of drugs,” Arch. Pharm. Chemi 86(1): 1-39 (1979); Bundgaard H. “Improved drug delivery by the prodrug approach,” Controlled Drug Delively 17: 179-96 (1987); Bundgaard H. “Prodrugs as a means to improve the delivery of peptide drugs”,Arfv. Drug Delivel)' Rev. 8(1): 1-38 (1992); Fleisher et al. “Improved oral drug delivery: solubility limitations overcome by the use of prodrugs,” Arfv. Drug Delivery Rev. 19(2): 115-130 (1996); Fleisher et al. “Design of prodrugs for improved gastrointestinal absorption by intestinal enzyme targeting,” Methods Enzymol. 112 (Drug Enzyme Targeting, Pt. A): 360-81, (1985); Farquhar D, et al., “Biologically Reversible Phosphate-Protective Groups,” Pharm. Sci., 72(3): 324-325 (1983); Freeman S, et al., “Bioreversible Protection for the Phospho Group: Chemical Stability and Bioactivation of Di(4-acetoxybenzyl) Methylphosphonate with Carboxyesterase,” Chern. Soc., Chern. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups,” Eur. J. Pharm. Sci. 4: 49-59 (1996); Gangwar et al., “Pro-drug, molecular structure and percutaneous delivery,” Des. Biophamz. Prop. Prodrugs Analogs, [Symp.] Meeting Date 1976, 409-21. (1977); Nathwani and Wood, “Penicillins: a current review of their clinical pharmacology and therapeutic use,” Drugs 45(6): 866-94 (1993); Sinhababu and Thakker, “Prodrugs of anticancer agents,” Adv. Drug Delivery Rev. 19(2): 241-273 (1996); Stella et al., “Prodrugs. Do they have advantages in clinical practice?,” Drugs 29(5): 455-73 (1985); Tan et al. “Development and optimization of anti-HIV nucleoside analogs and prodrugs: A review of their cellular pharmacology, structure-activity relationships and pharmacokinetics” Adv. Drug Deliva}' Rev. 39(1-3): 117-151 (1999); Taylor, “Improved passive oral drug delivery via prodrugs,” Adv. Drug Delivery Rev., 19(2): 131-148 (1996); Valentino and Borchardt, “Prodrug strategies to enhance the intestinal absorption of peptides,” Drug Discovery Today 2(4): 148-155 (1997); Wiebe and Knaus, “Concepts for the design of anti-HIV nucleoside prodrugs for treating cephalic HIV infection,” Adv. Drug Delivery Rev.: 39(1-3):63-80 (1999); Waller et al., “Prodrugs,” Br. J. Clin. Pharmac. 28: 497-507 (1989), which are incorporated by reference herein in their entireties.

In some embodiments, a GDF11 polypeptide as described herein can be a pharmaceutically acceptable solvate. The term “solvate” refers to a peptide as described herein in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The peptides of the present invention can be synthesized by using well known methods including recombinant methods and chemical synthesis. Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology: Peptide Synthesis Protocols, Vol 35, Humana Press, Totawa, N.J. (1994), contents of both of which are herein incorporated by reference. Peptides can also be chemically synthesized using methods well known in the art. See for example, Merrifield et al., J. Am. Chern. Soc. 85:2149 (1964); Bodanszky, M., Principles of Peptide Synthesis, Springer-Verlag, New York, N.Y. (1984); Kirnrnerlin, T. and Seebach, D. J. Pept. Res. 65:229-260 (2005); Nilsson et al., Annu. Rev. Biophys. Biornol. Struct. (2005) 34:91-118; W. C. Chan and P. D. White (Eds.) Frnoc Solid Phase Peptide Synthesis: A Practical Approach, Oxford University Press, Cary, N.C. (2000); N. L. Benoiton, Chemistry of Peptide Synthesis, CRC Press, Boca Raton, Fla. (2005); J. Jones, Amino Acid and Peptide Synthesis, 2nd Ed, Oxford University Press, Cary, N.C. (2002); and P. Lloyd-Williams, F. Albericio, and E. Giralt, Chemical Approaches to the synthesis of pep tides and proteins, CRC Press, Boca Raton, Fla. (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

In some embodiments, the technology described herein relates to a nucleic acid encoding a GDF11 polypeptide as described herein. As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double stranded DNA. Alternatively, it can be a single-stranded nucleic acid not derived from any double stranded DNA. In one aspect, the template nucleic acid is DNA. In another aspect, the template is RNA. Suitable nucleic acid molecules are DNA, including genomic DNA or cDNA. Other suitable nucleic acid molecules are RNA, including mRNA. The nucleic acid molecule can be naturally occurring, as in genomic DNA, or it may be synthetic, i.e., prepared based upon human action, or may be a combination of the two. The nucleic acid molecule can also have certain modification such as 2′-deoxy, 2′-deoxy-2′fluoro, 2′-0-methyl, 2′-0-methoxyethyl (2′-0-MOE), 2′-0-aminopropyl (2′-0-AP), 2′-0-dimethylaminoethyl (2′-0-DMAOE), 2′-0-dimethylaminopropyl (2′-0-DMAP), 2′-0-dimethylaminoethyloxyethyl (2′-0-DMAEOE), or 2′-0-N-methylacetamido (2′-0-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

In some embodiments, a nucleic acid encoding a GDF11 polypeptide can comprise a nucleotide sequence encoding SEQ ID NO: 3. In some embodiments, a nucleic acid encoding a GDF11 polypeptide as described herein is comprised by a vector. In some of the aspects described herein, a nucleic acid sequence encoding a GDF11 polypeptide as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a GDF11 polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In some embodiments the level of GDF11 in the subject is increased by at least 20% over the level of GDF11 in the subject prior to treatment, e.g. 20% or more, 30% or more, 40% or more, 50% or more, 100% or more, 150% or more, 200% or more, 250% or more, 300% or more, or 350% or more. In some embodiments the level of GDF11 in the subject is increased by at least 100% over the level of GDF11 in the subject prior to treatment. In some embodiments the level of GDF11 in the subject is increased by at least 200% over the level of GDF11 in the subject prior to treatment. In some embodiments the level of GDF11 in the subject is increased by about 250% over the level of GDF11 in the subject prior to treatment. In some embodiments, the level of GDF11 in the subject is increased to at least 50% of a healthy reference level, e.g. 50% or more, 60% or more, 70% or more, 80% or more, 90% or more, or 100% or more of a healthy reference level. In some embodiments, the level of GDF11 in the subject is increased to at least 60% of a healthy reference level. In some embodiments, the level of GDF11 in the subject is increased to at least 75% of a healthy reference level. In some embodiments, the level of GDF11 in the subject is increased to at least 90% of a healthy reference level. A healthy reference level can be the average level of GDF11 in a population of human subjects (e.g., young individuals) not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis.

In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 70. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 65. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 60. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 55. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 50. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 45. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 40. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 35. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 30. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 25. In some embodiments, a healthy reference level can be the average level of GDF11 in a population of human subjects not exhibiting any signs or symptoms of neuromuscular junction fragmentation, degeneration, or deterioration and related disorders, motor neuron degeneration and related disorders, skeletal muscle conditions, such as muscle atrophy, and neuromuscular diseases, such as amyotrophic lateral sclerosis, and who are under the age of 20.

In some embodiments, the methods described herein can comprise selecting a subject with a level of GDF11 which is lower than a healthy reference level and administering a treatment as described herein.

In some embodiments, the level of GDF11 in a subject is increased in order to rejuvenate neuromuscular junctions in a subject in need thereof.

In some embodiments, the level of GDF11 in a subject is increased in order to treat neuromuscular junction fragmentation and related disorders in a subject in need thereof. In some embodiments, the level of GDF11 in a subject is increased in order to prevent neuromuscular junction fragmentation and related disorders in a subject in need thereof. In some embodiments, the level of GDF11 in a subject is increased in order to delay the onset of neuromuscular junction fragmentation and related disorders in a subject in need thereof.

In some embodiments, the level of GDF11 in a subject is increased in order to treat neuromuscular junction degeneration and related disorders in a subject in need thereof. In some embodiments, the level of GDF11 in a subject is increased in order to prevent neuromuscular junction degeneration and related disorders in a subject in need thereof. In some embodiments, the level of GDF11 in a subject is increased in order to delay the onset of neuromuscular junction degeneration and related disorders in a subject in need thereof.

In some embodiments, the level of GDF11 in a subject is increased in order to treat motor neuron degeneration and related disorders in a subject in need thereof. In some embodiments, the level of GDF11 in a subject is increased in order to prevent motor neuron degeneration and related disorders in a subject in need thereof. In some embodiments, the level of GDF11 in a subject is increased in order to delay the onset of motor neuron degeneration and related disorders in a subject in need thereof.

In some embodiments, the level of GDF11 in a subject is increased in order to treat muscle atrophy and related disorders in a subject in need thereof. In some embodiments, the level of GDF11 in a subject is increased in order to prevent muscle atrophy and related disorders in a subject in need thereof. In some embodiments, the level of GDF11 in a subject is increased in order to delay the onset of muscle atrophy and related disorders in a subject in need thereof.

In some embodiments, the level of GDF11 in a subject is increased in order to treat neuromuscular disease in a subject in need thereof. In some embodiments, the level of GDF11 in a subject is increased in order to prevent a neuromuscular disease in a subject in need thereof. In some embodiments, the level of GDF11 in a subject is increased in order to delay the onset of a neuromuscular disease in a subject in need thereof.

In some embodiments, the level of GDF11 in a subject is increased in order to treat amyotrophic lateral sclerosis or a symptom associated with amyotrophic lateral sclerosis in a subject in need thereof. In some embodiments, the level of GDF11 in a subject is increased in order to prevent amyotrophic lateral sclerosis in a subject in need thereof. In some embodiments, the level of GDF11 in a subject is increased in order to delay the onset of amyotrophic lateral sclerosis in a subject in need thereof.

Neuromuscular junction fragmentation, degeneration, or deterioration and related disorders, motor neuron degeneration and related disorders, skeletal muscle conditions (e.g., muscle atrophy), and neuromuscular diseases (e.g., amyotrophic lateral sclerosis) related to low or decreased GDF11 polypeptide tend to develop with the decrease in GDF11 levels that occur with increasing age. Thus, it is expected that such conditions can be prevented or, at a minimum, delayed, by maintaining GDF11 polypeptide levels at or near the level found in normal, healthy young adults, e.g. by administering a GDF11 polypeptide or a nucleic acid encoding a GDF11 polypeptide with advancing age, but prior to the onset of such conditions, diseases, or disorders.

Aspects of the technology described herein relate to compositions comprising a GDF11 polypeptide as described herein or a nucleic acid encoding a GDF11 polypeptide as described herein. In some embodiments, the composition is a pharmaceutical composition. As used herein, the term “pharmaceutical composition” refers to the active agent in combination with a pharmaceutically acceptable carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In some aspects, a composition described herein comprising a GDF11 polypeptide or functional fragment or variant thereof can be used for rejuvenating neuromuscular junctions in a subject in need thereof, wherein increased levels of the GDF11 polypeptide or functional fragment or variant thereof in the subject rejuvenate neuromuscular junctions in the subject.

In some aspects, a composition described herein comprising a GDF11 polypeptide or a functional fragment or variant thereof can be used for treating, preventing, or delaying the onset of, neuromuscular junction fragmentation and related disorders in a subject in need thereof, wherein increased levels of the GDF11 polypeptide or functional fragment or variant thereof in the subject reduce or reverse neuromuscular junction fragmentation in the subject, thereby treating, preventing, or delaying the onset of, neuromuscular junction fragmentation and related disorders in the subject.

In some aspects, a composition described herein comprising a GDF11 polypeptide or a functional fragment or variant thereof can be used for treating, preventing, or delaying the onset of neuromuscular junction degeneration or a related disorder in a subject in need thereof, wherein increased levels of the GDF11 polypeptide or functional fragment or variant thereof treat, prevent, or delay the onset of, neuromuscular junction degeneration or the related disorder in the subject.

In some aspects, a composition comprising a GDF11 polypeptide or a functional fragment or variant thereof can be used for treating, preventing, or delaying the onset of, motor neuron degeneration or a related disorder in a subject in need thereof, wherein increased levels of the GDF11 polypeptide or functional fragment or variant thereof treat, prevent, or delay the onset of the motor neuron degeneration or the related disorder in the subject.

In some aspects, a composition comprising a GDF11 polypeptide or a functional fragment or variant thereof can be used for treating, preventing, or delaying the onset of, muscle atrophy in a subject in need thereof, wherein increased levels of the GDF11 polypeptide or functional fragment or variant thereof treat, prevent, or delay the onset of muscle atrophy in the subject.

In some aspects, a composition comprising a GDF11 polypeptide or a functional fragment or variant thereof can be used for treating, preventing, or delaying the onset of, neuromuscular disease in a subject in need thereof, wherein increased levels of the GDF11 polypeptide or functional fragment or variant thereof treat, prevent, or delay the onset of the neuromuscular disease.

In some aspects, a composition comprising a GDF11 polypeptide or a functional fragment or variant thereof can be used for treating, preventing, or delaying the onset of, amyotrophic lateral sclerosis in a subject in need thereof, wherein increased levels of the GDF11 polypeptide or functional fragment or variant thereof treat, prevent, or delay the onset of amyotrophic lateral sclerosis in the subject.

In certain embodiments, the inventions disclosed herein increase the concentration or production of endogenous GDF11 in a subject. For example, in certain aspects, the concentration of GDF11 polypeptide in a subject (e.g., the concentration of GDF11 in the skeletal muscle of a subject) may be increased by exercise. Accordingly, in certain embodiments the inventions disclosed herein relate to methods of rejuvenating neuromuscular junctions in a subject in need thereof, comprising increasing the level of GDF11 polypeptide in the subject by exposing such subject to an exercise regimen. Also disclosed are methods of treating, preventing, or delaying the onset of neuromuscular junction fragmentation or a related disorder in a subject in need thereof, the method comprising increasing the level of GDF11 polypeptide in the subject by exposing such subject to an exercise regimen (e.g., an exercise regimen capable of building the size or strength of the subject's muscle tissue). Similarly, also disclosed are methods of treating, preventing, or delaying the onset of, muscle atrophy, neuromuscular junction degeneration, motor neuron degeneration or a neuromuscular disease (e.g., amyotrophic lateral sclerosis) in a subject in need thereof, the method comprising increasing the level of GDF11 polypeptide in the subject by exposing such subject to an exercise regimen.

In some embodiments of this and other aspects described herein, the subject has been diagnosed with a disease, condition, or disorder described herein due to aging.

In some embodiments, the composition causes in the subject one or more of a decrease in fragmentation of neuromuscular junctions, an increase or maintenance of neuromuscular innervation, a decrease of neuromuscular denervation, preservation or restoration of motor units, an increase in the size of postsynaptic endplates, an increases in the number of postsynaptic endplates, an increase in the length of postsynaptic endplates, an increase in the density of postsynaptic folds, normalization of neuromuscular junction morphology, a decrease in denervation of fast-twitch fibers, an increase in the number of nerve terminal branches per endplate, an increase in the number of terminal sprouts, an increase or maintenance of synaptic vesicles, mitochondrial content, or nerve terminal area in presynaptic terminals, reversal of age-related changes in the quantal content of neurotransmitter release measured by decreases in amplitude of evoked endplate potentials (EPP), a reversal of age-related decline in axonal transport, preservation or restoration of muscle fibers, an increase in acetylcholine receptor number, a decrease in partially innervated or completed denervated neuromuscular junctions, an increase in the number of functional motor units in fast twitch muscle, enhanced neuromuscular recovery, enhanced physical performance, an increase in neuromuscular junction area, an increase in muscle size, and any combination thereof.

A still further aspect of the invention provides a pharmaceutical composition or kit of parts according to the invention for use in rejuvenating neuromuscular junctions. A still further aspect of the invention provides a pharmaceutical composition or kit of parts according to the invention for use in treating, preventing, or delaying the onset of neuromuscular junction fragmentation or a related disorder. Another aspect of the invention provides a pharmaceutical composition or kit of parts according to the invention for use in treating, preventing, or delaying the onset of, neuromuscular junction degeneration or a related disorder. Another aspect of the invention provides a pharmaceutical composition or kit of parts according to the invention for use in treating, preventing, or delaying the onset of, motor neuron degeneration or a related disorder. Another aspect of the invention provides a pharmaceutical composition or kit of parts according to the invention for use in treating, preventing, or delaying the onset of, muscle atrophy or a related disorder. Another aspect of the invention provides a pharmaceutical composition or kit of parts according to the invention for use in treating, preventing, or delaying the onset of, neuromuscular disease. Another aspect of the invention provides a pharmaceutical composition or kit of parts according to the invention for use in treating, preventing, or delaying the onset of, amyotrophic lateral sclerosis.

The preparation of a pharmacological composition that contains active ingredients dissolved or dispersed therein is well understood in the art and generally need not be limited based on formulation. Typically such compositions are prepared as injectable either as liquid solutions or suspensions, however, solid forms suitable for solution, or suspension, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents, and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine, and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline.

Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition will depend on the nature of the disorder or condition, and can be determined by standard clinical techniques.

In some embodiments, a GDF11 polypeptide or nucleic acid encoding a GDF11 polypeptide as described herein can be administered by controlled- or delayed-release means. Controlled release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Chemg-ju, Controlled-release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like.

Advantageously, controlled-release formulations can be used to control a drug's onset of action, duration of action, plasma levels within the therapeutic window, and peak blood levels. In particular, controlled or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B 1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments, the technology described herein relates to a syringe comprising a therapeutically effective amount of a composition e.g. a pharmaceutical preparation comprising a GDF11 polypeptide as described herein.

As used herein, the phrase “therapeutically effective amount,” “effective amount,” or “effective dose” refers to an amount that provides a therapeutic or aesthetic benefit in the treatment, prevention, or management of, for example, neuromuscular junction deterioration, fragmentation, or degeneration and related disorders, motor neuron degeneration and related disorders, skeletal muscle conditions (e.g., muscle atrophy), neuromuscular disease (e.g., amyotrophic lateral sclerosis).

Determination of a therapeutically effective amount is well within the capability of those skilled in the art; Generally, a therapeutically effective amount can vary with the subject's history, age, condition, sex, as well as the severity and type of the medical condition in the subject, and administration of other pharmaceutically active agents.

In one aspect, the technology described herein relates to a method comprising administering a GDF11 polypeptide or a nucleic acid encoding a GDF11 polypeptide to a subject. In some embodiments, the methods further comprise administering a neurotrophic factor, a myotrophic factor, a myogenic regulatory factor, or a combination thereof, to a subject. In some embodiments, the method is a method of treating a subject in need of treatment for a condition, disorder, or disease described herein. In some embodiments, the method is a method of preventing, or delaying the onset of, a condition, disorder, or disease described herein in a subject.

As used herein, “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of, for example, a condition, disease, or disorder described herein, or delaying or slowing onset of a condition, disease, or disorder described herein, and an increased lifespan as compared to that expected in the absence of treatment.

As used herein, the term “administering,” refers to the placement of the composition comprising a GDF11 polypeptide or a nucleic acid encoding a GDF11 polypeptide as disclosed herein into a subject by a method or route which results in delivery to a site of action. The pharmaceutical composition comprising a GDF11 polypeptide or a nucleic acid encoding a GDF11 polypeptide can be administered by any appropriate route which results in an effective treatment in the subject.

Data described herein indicate that systemic administration via the vascular system can be effective. Thus administration via the intravenous route is specifically contemplated. However, with appropriate formulation, other routes are contemplated, including, for example, intranasally, intraarterially; intra-coronary arterially; orally, by inhalation, intraperitoneally, intramuscularly, subcutaneously, intracavity, intrathecally, or by other means known by those skilled in the art. The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Therapeutic compositions containing at least one agent can be conventionally administered in a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The dosage ranges for the agent depends upon the potency, and are amounts large enough to produce the desired effect e.g., rejuvenation of a subject's neuromuscular junctions, or a reversal of neuromuscular junction fragmentation or related disorder, a reversal of neuromuscular junction degeneration or a related disorder, a reversal of motor neuron degeneration or a related disorder, a reversal of a skeletal muscle condition (e.g., muscle atrophy), a reversal of a neuromuscular disease (e.g., amyotrophic lateral sclerosis). The dosage should not be so large as to cause unacceptable adverse side effects.

Generally, the dosage will vary with the age, condition, and sex of the patient and can be determined by one of skill in the art. The dosage can also be adjusted by the individual physician in the event of any complication. Typically, the dosage can range from 0.001 mg/kg body weight to 0.5 mg/kg body weight. In one embodiment, the dose range is from 5 μg/kg body weight to 30 μg/kg body weight.

Administration of the doses recited above can be repeated. In some embodiments, the doses are given once a day, or multiple times a day, for example, but not limited to, three times a day. In some embodiments, the doses recited above are administered daily for weeks or months. The duration of treatment depends upon the subject's clinical progress and responsiveness to therapy. Without wishing to be bound by theory, where the GDF11 polypeptide apparently diminishes with age in affected individuals, it is expected that long-term therapy would be required to establish and maintain the benefit of GDF11-based treatment, e.g. rejuvenating neuromuscular junctions, or treating, preventing, or delaying the onset of, neuromuscular junction fragmentation or degeneration and related disorders, motor neuron degeneration and related disorders, skeletal muscle conditions (e.g., muscle atrophy), and neuromuscular diseases (e.g., amyotrophic lateral sclerosis).

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent administration. Alternatively, continuous intravenous infusion sufficient to maintain concentrations in the blood in the ranges specified for in vivo therapies are contemplated. In some embodiments, the dosage range is sufficient to maintain concentrations in the blood in the range found in the blood of a population of normal, healthy human subjects (e.g. those with no signs, symptoms, or makers of neuromuscular junction fragmentation, degeneration, or deterioration) under the age of 50. In some embodiments, the dosage range is sufficient to maintain concentrations in the blood in the range found in normal, healthy human subjects under the age of 40. In some embodiments, the dosage range is sufficient to maintain concentrations in the blood in the range found in normal, healthy human subjects under the age of 30.

A therapeutically effective amount is an amount of an agent that is sufficient to produce a statistically significant, measurable change in, for example, reversal of neuromuscular junction fragmentation or a related disorder, reversal of neuromuscular junction degeneration or a related disorder, reversal of motor neuron degeneration or a related disorder, reversal of a skeletal muscle condition (e.g., muscle atrophy), or reversal of a neuromuscular disease (e.g., amyotrophic lateral sclerosis). Such effective amounts can be gauged in clinical trials as well as animal studies. Efficacy of an agent can be determined by assessing physical indicators of, for example neuromuscular junction deterioration as described above herein. In experimental systems, assays for efficacy include measurement of skeletal muscle mass as well as, determination of myofiber size as determined by histological microscopy, and/or an improvement in neuromuscular junction morphology. Such assays are well known in the art and described in detail herein. Clinically acceptable methods for detecting or monitoring neuromuscular junction rejuvenation are apparent from the teachings described herein. In addition, efficacy of an agent can be measured by an increase in GDF11 polypeptides or fragments thereof in a subject being treated with an agent comprising a GDF11 polypeptide or a nucleic acid encoding GDF11 polypeptide.

The efficacy of a given treatment for a condition, disease, or disorder described herein (e.g., amyotrophic lateral sclerosis) can be determined by the skilled clinician. However, a treatment is considered “effective treatment,” as the term is used herein, if any one or all of the signs or symptoms of e.g., amyotrophic lateral sclerosis are altered in a beneficial manner, other clinically accepted symptoms are improved or ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein.

In some embodiments, the methods further comprise administering the pharmaceutical composition described herein along with one or more additional agents, biologics, drugs, or treatments beneficial to a subject suffering from neuromuscular junction fragmentation or related disorder, a neuromuscular junction degeneration or a related disorder, motor neuron degeneration or a related disorder, a skeletal muscle condition (e.g., muscle atrophy), a neuromuscular disease (e.g., amyotrophic lateral sclerosis), as part of a combinatorial therapy. In some such embodiments, the agent, biologic, drug, or treatment can be selected from the group consisting of: modulators of one or more of skeletal myosin, skeletal actin, skeletal tropomyosin, skeletal troponin C, skeletal troponin I, skeletal troponin T, and skeletal muscle, including fragments and isoforms thereof, and the skeletal sarcomere and other suitable therapeutic agents useful in the treatment of the aforementioned diseases including: anti-obesity agents, anti-sarcopenia agents, anti-wasting syndrome agents, anti-frailty agents, anti-cachexia agents, anti-muscle spasm agents, agents against post-surgical and post-traumatic muscle weakness, and anti-neuromuscular disease agents, as well as the agents described in U.S. Patent Application No. 2005/0197367.

In some such embodiments, the agent, biologic, drug, or treatment comprises an agent that promotes survival and maintenance of presynaptic and postsynaptic apparatus at the neuromuscular junction. In some embodiments, the agent is selected from the group consisting of a neurotrophic factor, a myotrophic factor, a myogenic regulatory factor, and combinations thereof.

Examples of neurotrophic factors include neurotrophins, glial cell-line derived neurotrophic factor family ligands (GFLs), cytokines, and growth factors. Suitable neurotrophins include, for example, nerve growth factor (NGF or beta-NGF) or a functional fragment or variant thereof, brain-derived neurotrophic factor (BDNF) or a functional fragment or variant thereof, neurotrophin-3 (NT-3) or a functional fragment or variant thereof, and neurotrophin-4 or a functional fragment or variant thereof. BDNF, NT-3 and NT-4 are known to be involved in maintaining acetylcholine receptor clustering in the neuromuscular junction. Suitable GFLs include, for example, glial cell line-derived neurotrophic factor (GDNF) or a functional fragment or variant thereof, neurturin (NRTN) or a functional fragment or variant thereof, artemin (ARTN) or a functional fragment or variant thereof, and persephin (PSPN) or a functional fragment or variant thereof. Suitable cytokines include GDNF, ciliary neurotrophin factor (CNTF) or a functional fragment or variant thereof.

An exemplary myotrophic factor for use in the methods and compositions of the present invention include, for example, CNTF, a pleiotropic cytokine.

Myogenic regulatory factors (MRFs) comprise a family of helix-loop-helix transcription factors which regulate muscle and neuromuscular junction regeneration. MRFs activate myoblast formation, satellite cell proliferation, and preserve the fast-twitch, IIB/IIX fiber type during differentiation and regeneration. MRFs also play a role in regulating neuromuscular junction subtype expression, and particularly are believed to play a critical role in promoting neuromuscular junction and muscle recovery. Exemplary myogenic regulatory factors include, for example, MyfS, MRF4, myogenin, and MyoD.

Exemplary growth factors which play a modulatory neuromuscular role during aging, and are useful in combination with the compositions of the present invention, include, for example, insulin-like growth factor (IGF-1 and IGF-II), fibroblast growth factors (FGFs), and epidermal growth factors (EGF). An exemplary EGF family protein includes, for example, neuregulin, which binds to its cognate receptors in postsynaptic muscle fibers and results in clustering of acetylcholine receptor and transcription of proteins responsible for maintaining synaptic transmission and associated apparatus.

Suitable additional medicinal and pharmaceutical agents include, for example: orlistat, sibramine, diethylpropion, phentermine, benzaphetamine, phendimetrazine, estrogen, estradiol, levonorgestrel, norethindrone acetate, estradiol valerate, ethinyl estradiol, norgestimate, conjugated estrogens, esterified estrogens, medroxyprogesterone acetate, insulin-derived growth factor, human growth hormone, riluzole, cannabidiol, prednisone, beta agonists (e.g., albuterol), myostatin inhibitors, selective androgen receptor modulators, non-steroidal anti-inflammatory drugs, and botulinum toxin.

Other suitable medicinal and pharmaceutical agents include TRH, diethylstilbesterol, theophylline, enkephalins, E series prostaglandins, compounds disclosed in U.S. Pat. No. 3,239,345 (e.g., zeranol), compounds disclosed in U.S. Pat. No. 4,036,979 (e.g., sulbenox), peptides disclosed in U.S. Pat. No. 4,411,890 growth hormone secretagogues such as GHRP-6, GHRF-1 (disclosed in U.S. Pat. No. 4,411,890 and publications WO 89/07110 and WO 89/07111), GHRP-2 (disclosed in WO 93/04081), NN703 (Novo Nordisk), LY444711 Lilly), MK-677 (Merck), CP424391 (Pfizer) and B-HT920, growth hormone releasing factor and its analogs, growth hormone and its analogs and somatomedins including IGF-1 and IGF-2, leukemia inhibitory factor, cilia neurotrophic factor, brain derived neurotrophic factor, interleukin 6, interleukin 15, alpha-adrenergic agonists, such as clonidine or serotonin 5-HT_{D} agonists, such as sumatriptan, agents which inhibit somatostatin or its release, such as physostigmine, pyridostigmine, parathyroid hormone, PTH(1-34), and bisphosphonates, such as MK-217 (alendronate).

Still other suitable medicinal and pharmaceutical agents include estrogen, testosterone, selective estrogen receptor modulators, such as tamoxifen or raloxifene, other androgen receptor modulators, such as those disclosed in Edwards, J. P. et. al., Bio. Med. Chem. Let., 9, 1003-1008 (1999) and Hamann, L. G. et. al., J. Med. Chem., 42, 210-212 (1999), and progesterone receptor agonists (“PRA”), such as levonorgestrel, medroxyprogesterone acetate (MPA).

Still other suitable medicinal and pharmaceutical agents include aP2 inhibitors, such as those disclosed in U.S. Ser. No. 09/519,079 filed Mar. 6, 2000, PPAR gamma antagonists, PPAR delta agonists, beta 2 adrenergic agonists, beta 3 adrenergic agonists, such as AJ9677 (Takeda/Dainippon), L750355 (Merck), or CP331648 (Pfizer), other beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), a thyroid receptor beta drug, such as a thyroid receptor ligand as disclosed in WO 97/21993, WO 99/00353, and GB98/284425, and anorectic agents, such as dexamphetamine, phentermine, phenylpropanolamine or mazindol.

Still other suitable medicinal and pharmaceutical agents include HIV and AIDS therapies, such as indinavir sulfate, saquinavir, saquinavir mesylate, ritonavir, lamivudine, zidovudine, lamivudine/zidovudine combinations, zalcitabine, didanosine, stavudine, and megestrol acetate.

Still other suitable medicinal and pharmaceutical agents include antiresorptive agents, hormone replacement therapies, vitamin D analogues, elemental calcium and calcium supplements, cathepsin K inhibitors, MMP inhibitors, vitronectin receptor antagonists, Src SH₂ antagonists, vacular —H⁺-ATPase inhibitors, ipriflavone, fluoride, Tibo lone, pro stanoids, 17-beta hydroxysteroid dehydrogenase inhibitors and Src kinase inhibitors.

The above other therapeutic agents, when employed in combination with the chemical entities described herein, may be used, for example, in those amounts indicated in the Physicians' Desk Reference (PDR) or as otherwise determined by one of ordinary skill in the art.

It should be noted that while the inventions disclosed herein generally relate to pharmacological interventions (e.g., the administration of compositions comprising a GDF11 polypeptide to a subject having ALS), the inventions disclosed herein are not limited to pharmacological interventions. In certain embodiments, the present inventions also relate to the findings that levels of GDF11 in a subject may be influenced (e.g., increased) by non-pharmacologic interventions. For example, endogenous concentrations of GDF may be increased in a subject with diet and exercise. In certain embodiments, a subject's levels or concentrations of GDF11 polypeptide (e.g., the concentration of GDF11 polypeptide in the skeletal muscle of a subject) may be increased by exercise and the administration of a high fat diet to the subject. In certain aspects, disclosed herein are methods of rejuvenating neuromuscular junctions in a subject in need thereof, comprising increasing the level of GDF11 polypeptide in the subject by exposing such subject to an exercise regimen and a high fat diet. Also disclosed are methods of treating, preventing, or delaying the onset of, neuromuscular junction fragmentation or a related disorder in a subject in need thereof, the method comprising increasing the level of GDF11 polypeptide in the subject by exposing such subject to an exercise regimen and a high fat diet. Similarly, also disclosed are methods of treating, preventing, or delaying the onset of, muscle atrophy, neuromuscular junction degeneration, motor neuron degeneration or a neuromuscular disease (e.g., amyotrophic lateral sclerosis) in a subject in need thereof, the method comprising increasing the level of GDF polypeptide in the subject by exposing such subject to an exercise regimen (e.g., an exercise regimen capable of building the size or strength of the subject's muscle tissue) and a high fat diet.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately,” the invention includes an embodiment in which the exact value is recited. For any embodiment of the invention in which a numerical value is not prefaced by “about” or “approximately,” the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

What is claimed is:

1. A method of rejuvenating neuromuscular junctions in a subject in need thereof, comprising administering to the subject a composition which increases the level of GDF11 polypeptide in the subject.

2. A method of treating, preventing, or delaying the onset of, neuromuscular junction fragmentation or a related disorder in a subject in need thereof, the method comprising administering to the subject an effective amount of a composition which increases the level of GDF11 polypeptide in the subject.

3. A method of treating, preventing, or delaying the onset of, neuromuscular junction degeneration or a related disorder in a subject in need thereof, the method comprising administering to the subject an effective amount of a composition which increases the level of GDF11 polypeptide in the subject.

4. A method of treating, preventing, or delaying the onset of, motor neuron degeneration or a related disorder in a subject in need thereof, the method comprising administering to the subject an effective amount of a composition which increases the level of GDF11 polypeptide in the subject.

5. A method of treating, preventing, or delaying the onset of, muscle atrophy in a subject in need thereof, the method comprising administering to the subject an effective amount of a composition which increases the level of GDF11 polypeptide in the subject.

6. A method of treating, preventing, or delaying the onset of, a neuromuscular disease in a subject in need thereof, the method comprising administering to the subject an effective amount of a composition which increases the level of GDF11 polypeptide in the subject.

7. A method of treating, preventing, or delaying the onset of amyotrophic lateral sclerosis in a subject in need thereof, the method comprising administering to the subject an effective amount of a composition which increases the level of GDF11 polypeptide in the subject.

8. The method of any one of claims 1-7, wherein the composition causes one or more of a decrease in fragmentation of neuromuscular junctions, an increase or maintenance of neuromuscular innervation, a decrease of neuromuscular denervation, preservation or restoration of motor units, an increase in the size of postsynaptic endplates, an increases in the number of postsynaptic endplates, an increase in the length of postsynaptic endplates, an increase in the density of postsynaptic folds, normalization of neuromuscular junction morphology, a decrease in denervation of fast-twitch fibers, an increase in the number of nerve terminal branches per endplate, an increase in the number of terminal sprouts, an increase or maintenance of synaptic vesicles, mitochondrial content, or nerve terminal area in presynaptic terminals, reversal of age-related changes in the quantal content of neurotransmitter release measured by decreases in amplitude of evoked endplate potentials (EPP), a reversal of age-related decline in axonal transport, preservation or restoration of muscle fibers, an increase in acetylcholine receptor number, a decrease in partially innervated or completed denervated neuromuscular junctions, an increase in the number of functional motor units in fast twitch muscle, enhanced neuromuscular recovery, enhanced physical performance, an increase in neuromuscular junction area, an increase in muscle size, and any combination thereof.

9. The method of any one of claims 1-8, the subject has been diagnosed with a condition, disease, or disorder associated with aging.

10. The method of claim 9, wherein the condition, disease, or disorder associated with aging is selected from the group consisting of a skeletal muscle condition, a neuromuscular disease, a neurodegenerative disorder, a neuromuscular junction disease, and combinations thereof.

11. The method of any one of claims 1-10, wherein the level of GDF11 polypeptide is increased in the systemic circulation of the subject.

12. The method of any one of claims 1-11, wherein the level of GDF11 polypeptide is increased in the skeletal muscle tissue of the subject.

13. The method of any one of claims 1-12, wherein the composition comprises an agonist antibody that increases expression or activity of GDF11 polypeptide in the subject.

14. The method of any one of claims 1-13, wherein the composition comprises an isolated or recombinant GDF11 polypeptide.

15. The method of any one of claims 1-14, wherein the composition comprises a GDF11 polypeptide comprising the amino acid sequence of SEQ ID NO: 3.

16. The method of any one of claims 1-14, wherein the composition comprises a GDF11 polypeptide comprising the amino acid sequence of SEQ ID NO: 2.

17. The method of any one of claims 1-14, wherein the composition comprises a GDF11 polypeptide comprising the amino acid sequence of SEQ ID NO: 1.

18. The method of any one of claims 1-14, wherein the composition comprises a GDF11 polypeptide comprising the amino acid sequence of SEQ ID NO: 4.

19. The method of any one of claims 1-18, wherein the composition comprises homodimers of GDF11 polypeptides comprising the amino acid sequence of any of SEQ ID NOs: 1, 2, 3 or 4.

20. The method of any one of claims 1-19, wherein the composition comprises complexes of GDF11 polypeptides comprising the amino acid sequence of any of SEQ ID NOs: 1, 2, 3 or 4.

21. The method of any one of claims 1-20, wherein the composition comprises a nuclei acid encoding a GDF11 polypeptide or a functional fragment or variant thereof.

22. The method of any one of claims 1-21, wherein the GDF11 polypeptide comprises a modified GDF11 polypeptide.

23. The method of claim 22, wherein the modified GDF11 polypeptide comprises a modification selected from the group consisting of fusion to an Fe fragment, pegylation, conjugation to albumin, an amino acid mutation that prevents or reduces proteolytic degradation, an amino acid mutation that prolongs half-life, and any combination thereof.

24. The method of any one of claims 1-23, wherein the composition is administered via a route selected from the group consisting of intravenously, subcutaneously, intra-arterially, and intra-muscularly, and intrathecally.

25. The method of any one of claims 1-24, wherein the level of GDF11 polypeptide is increased by at least 100%.

26. The method of any one of claims 1-25, wherein the level of GDF11 polypeptide is increased to at least 75% of a healthy reference level.

27. A pharmaceutical composition comprising a GDF11 polypeptide or a functional fragment or variant thereof, and a pharmaceutically acceptable carrier.

28. The pharmaceutical composition of claim 27, further comprising an agent that promotes survival and maintenance of presynaptic and postsynaptic apparatus at the neuromuscular junction.

29. The pharmaceutical composition of any one of claims 27-28, wherein the agent is selected from the group consisting of a neurotrophic factor, a myotrophic factor, a myogenic regulatory factor, and combinations thereof.

30. The use of a composition comprising a GDF11 polypeptide or functional fragment or variant thereof for rejuvenating neuromuscular junctions in a subject in need thereof, wherein increased levels of the GDF11 polypeptide or functional fragment or variant thereof in the subject rejuvenate neuromuscular junctions in the subject.

31. The use of a composition comprising a GDF11 polypeptide or a functional fragment or variant thereof for treating, preventing, or delaying the onset of neuromuscular junction fragmentation or a related disorder in a subject in need thereof, wherein increased levels of the GDF11 polypeptide or functional fragment or variant thereof reverses or reduces neuromuscular junction fragmentation in the subject, thereby treating, preventing, or delaying the onset of, neuromuscular junction fragmentation or the related disorder in the subject.

32. The use of a composition comprising a GDF11 polypeptide or a functional fragment or variant thereof for treating, preventing, or delaying the onset of, neuromuscular junction degeneration or a related disorder in a subject in need thereof, wherein increased levels of the GDF11 polypeptide or functional fragment or variant thereof in the subject treat, prevent, or delay the onset of, neuromuscular junction degeneration or the related disorder in the subject.

33. The use of a composition comprising a GDF11 polypeptide or a functional fragment or variant thereof for treating, preventing, or delaying the onset of, motor neuron degeneration or a related disorder in a subject in need thereof, wherein increased levels of the GDF11 polypeptide or functional fragment or variant thereof in the subject treat, prevent, or delay the onset of, motor neuron degeneration or the related disorder in the subject.

34. The use of a composition comprising a GDF11 polypeptide or a functional fragment or variant thereof for treating, preventing, or delaying the onset of, muscle atrophy in a subject in need thereof, wherein increased levels of the GDF11 polypeptide or functional fragment or variant thereof in the subject treat, prevent, or delay the onset of, muscle atrophy in the subject.

35. The use of a composition comprising a GDF11 polypeptide or a functional fragment or variant thereof for treating, preventing, or delaying the onset of, amyotrophic lateral sclerosis in a subject in need thereof, wherein increased levels of the GDF11 polypeptide or functional fragment or variant thereof in the subject treat, prevent, or delay the onset of, amyotrophic lateral sclerosis in the subject.

36. The use according to any one of claims 30-35, wherein the composition causes one or more of a decrease in fragmentation of neuromuscular junctions, an increase or maintenance of neuromuscular innervation, a decrease of neuromuscular denervation, preservation or restoration of motor units, an increase in the size of postsynaptic endplates, an increases in the number of postsynaptic endplates, an increase in the length of postsynaptic endplates, an increase in the density of postsynaptic folds, normalization of neuromuscular junction morphology, a decrease in denervation of fast-twitch fibers, an increase in the number of nerve terminal branches per endplate, an increase in the number of terminal sprouts, an increase or maintenance of synaptic vesicles, mitochondrial content, or nerve terminal area in presynaptic terminals, reversal of age-related changes in the quantal content of neurotransmitter release measured by decreases in amplitude of evoked endplate potentials (EPP), a reversal of age-related decline in axonal transport, preservation or restoration of muscle fibers, an increase in acetylcholine receptor number, a decrease in partially innervated or completed denervated neuromuscular junctions, an increase in the number of functional motor units in fast twitch muscle, enhanced neuromuscular recovery, enhanced physical performance, an increase in neuromuscular junction area, an increase in muscle size, and any combination thereof.

37. The use according to any one of claims 30-36, the subject has been diagnosed with a condition, disease, or disorder associated with aging.

38. The use according to claim 37, wherein the condition, disease, or disorder associated with aging is selected from the group consisting of a skeletal muscle condition, a neuromuscular disease, a neurodegenerative disorder, a neuromuscular junction disease, and combinations thereof.

39. The use according to any one of claims 30-38, wherein the composition increases the level of GDF11 polypeptide in the systemic circulation of the subject.

40. The use according to any one of claims 30-39, wherein the composition increases the level of GDF11 polypeptide in the skeletal muscle tissue of the subject.

41. The use according to any one of claims 30-40, wherein the composition comprises an agonist antibody that increases expression or activity of GDF11 polypeptide in the subject.

42. The use according to any one of claims 30-41, wherein the composition comprises an isolated or recombinant GDF11 polypeptide.

43. The use according to any one of claims 30-42, wherein the GDF11 polypeptide comprises the amino acid sequence of SEQ ID NO: 3.

44. The use according to any one of claims 30-43, wherein the GDF11 polypeptide comprises the amino acid sequence of SEQ ID NO: 2.

45. The use according to any one of claims 30-43, wherein the GDF11 polypeptide comprises the amino acid sequence of SEQ ID NO: 1.

46. The use according to any one of claims 30-43, wherein the GDF11 polypeptide comprises the amino acid sequence of SEQ ID NO: 4.

47. The use according to any one of claims 30-46, wherein the composition comprises homodimers of GDF11 polypeptides comprising the amino acid sequence of any of SEQ ID NOs: 1, 2, 3 or 4.

48. The use according to any one of claims 30-47, wherein the composition comprises complexes of GDF11 polypeptides comprising the amino acid sequence of any of SEQ ID NOs: 1, 2, 3 or 4.

49. The use according to any one of claims 30-48, wherein the composition comprises a nuclei acid encoding the GDF11 polypeptide or functional fragment or variant thereof.

50. The use according to any one of claims 30-49, wherein the GDF11 polypeptide comprises a modified GDF11 polypeptide.

51. The use according to claim 50, wherein the modified GDF11 polypeptide comprises a modification selected from the group consisting of fusion to an Fc fragment, pegylation, conjugation to albumin, an amino acid mutation that prevents or reduces proteolytic degradation, an amino acid mutation that prolongs half-life, and any combination thereof.

52. The use according to any one of claims 30-51, wherein the composition is administered via a route selected from the group consisting of intravenously, subcutaneously, intra-arterially, intra-muscularly, and intrathecally.

53. The use according to any one of claims 30-52, wherein the composition increases the level of the GDF11 polypeptide by at least 100%.

54. The use according to any one of claims 30-53, wherein the composition increases the level of GDF11 polypeptide to at least 75% of a healthy reference level.

55. The method of claims 1-25, further comprising exposing the subject to an exercise regimen, wherein the exercise regimen increases the level of GDF11 in the subject.

56. The method of claim 55, further comprising administering a high-fat diet to the subject, wherein the high fat diet increases the level of GDF11 in the subject.

57. A method of rejuvenating neuromuscular junctions in a subject in need thereof, the method comprising exposing the subject to an exercise regimen which increases the level of GDF11 in the subject and thereby rejuvenates the neuromuscular junctions.

58. A method of treating, preventing, or delaying the onset of, a neuromuscular disease in a subject in need thereof, the method comprising exposing the subject to an exercise regimen which increases the level of GDF11 in the subject and thereby treats, prevents, or delays the onset of the neuromuscular disease.

59. A method of treating, preventing, or delaying the onset of, neuromuscular junction fragmentation, neuromuscular degeneration or a related disorder in a subject in need thereof, the method comprising exposing the subject to an exercise regimen which increases the level of GDF11 in the subject.

60. The method of claims 57-59, wherein the exercise regimen increases muscle mass or muscle strength.

61. The method of claims 57-59, further comprising administering a high fat diet to the subject.

62. The method of claims 57-59, further comprising administering the composition of claim 27 to the subject.
